(19) United States
(12) Patent Application Publication
(10) Pub. No.: US 2026/0090909 A1
(43) Pub. Date: Apr. 2, 2026

(54) FACE MASKS WITH THERAPEUTIC COOLING

(71) Applicant: SharkNinja Operating LLC, Needham, MA (US)

(72) Inventors: Hannah Elizabeth Clark, Paisley (GB); Olivia Louise Parsons, Boston, MA (US); Christopher W. Hedges, Hampshire (GB); Christina J. Crowley, London (GB); Ellen Catherine Padula, Brighton, MA (US)

(21) Appl. No.: 18/901,543

(22) Filed: Sep. 30, 2024

(51) Int. Cl. A61F 7/00; A61F 7/02

(52) U.S. Cl. CPC A61F 7/0085; A61F 7/02; A61F 2007/0003; A61F 2007/0004; A61F 2007/0225

(57) ABSTRACT

Various illustrative systems, devices, and methods for face masks are provided. In general, a face mask is configured to provide cooling therapy to a user wearing the face mask. In an exemplary implementation, the face mask includes a thermoelectric cooling device, such as a Peltier device, a thermoelectric cooler (TEC), or other thermoelectric cooling device. The thermoelectric cooling device is configured to generate heat through a thermoelectric effect where a heat flux is created at a junction of two different types of materials. The heat flux creates a cold area and a hot area. The cold area is configured to face toward the user's face to provide cool energy to the user's skin.

### FIELD

The present disclosure generally relates to face masks and more particularly to face masks with therapeutic cooling.

### BACKGROUND

Cooling can be applied to skin on a user's face for various health and/or aesthetic reasons. Face masks worn by a user may be used to apply the cooling. Some face masks include a Peltier cooler to generate the cooling delivered to the user's face. However, Peltier coolers also generate heat. The heat can interfere adversely with the cooling effect provided by the face mask.

Accordingly, there remains a need for improved devices, systems, and methods for face masks.

### SUMMARY

In general, systems, devices, and methods for face masks are provided.

In one aspect, a therapeutic device is provided that in one implementation includes a face mask configured to be worn on a face of a user. The face mask includes a first thermoelectric cooling device, a second thermoelectric cooling device, and a plurality of light emitting diodes (LEDs). The first thermoelectric cooling device is configured to be positioned under a first eye of the user, is configured to generate cooling configured to be applied to skin under the first eye of the user, and is configured to generate heat. The second thermoelectric cooling device is configured to be positioned under a second eye of the user, is configured to generate cooling configured to be applied to skin under the second eye of the user, and is configured to generate heat. The plurality of LEDs are configured to emit light toward and to skin on the face of the user with the user wearing the face mask.

The therapeutic device can vary in any number of ways. For example, the first and second thermoelectric cooling devices can each be a Peltier device.

For another example, the LEDs can each be configured to emit different wavelengths of light. Further, the different wavelengths include a first wavelength in a range of 450 nm to 495 nm, a second wavelength in a range of 620 nm to 740 nm, and a third wavelength in a range of 800 nm to 2500 nm. Further, the first light can be blue light, the second light can be red light, and the third light can be infrared (IR) light. Further, the LEDs can each be configured to emit the different wavelengths of light simultaneously.

For yet another example, the face mask can also include a first fan, a first heat sink, a second fan, and a second heat sink, the first fan can be upstream of the first thermoelectric cooling device and be configured to cause air flow in the face mask along a first air flow path to dissipate the heat generated by the first thermoelectric cooling device, the first heat sink can be located along the first air flow path downstream of the first fan, the second fan can be upstream of the second thermoelectric cooling device and be configured to cause air flow in the face mask along a second air flow path to dissipate the heat generated by the second thermoelectric cooling device, and the second heat sink can be located along the second air flow path downstream of the second fan. Further, first air entering the face mask can be configured to flow upward to the first fan and then laterally inward to the first heat sink, and second air entering the face mask can be configured to flow to the second fan and then laterally inward to the second heat sink. Further, the first air flowing laterally inward to the first heat sink can be in an opposite direction to the second air flowing laterally inward to the second heat sink; and/or the face mask can also include first internal ducting and second internal ducting, the first internal ducting extending toward the first fan and extending from the first heat sink and the second internal ducting extending toward the second fan and extending from the second heat sink.

For still another example, the therapeutic device can also include a control unit operably coupled to the face mask, and the control unit can be configured to control the plurality of lights, the cooling configured to be applied to skin under the first eye of the user, and the cooling configured to be applied to skin under the second eye of the user. Further, the control unit can be configured to receive an input from the user and to control, based on the input, the cooling configured to be applied to skin under the first eye of the user and the cooling configured to be applied to skin under the second eye of the user.

For yet another example, the face mask can also include a first air inlet through which air external to the face mask is configured to enter a first air flow path in the face mask, a first air outlet through which the air is configured to exit the first air flow path to exit the face mask, a second air inlet through which air external to the face mask is configured to enter second air flow path in the face mask, and a second air outlet through which the air is configured to exit the second air flow path to exit the face mask. Further, the first air flow path can be U-shaped, and the second air flow path can be U-shaped; the first air inlet, the first air outlet, the second air inlet, and the second air outlet can each be located at a bottom of the face mask; and/or the first and second air paths can together define a W shape.

For another example, the face mask can be air tight around a perimeter of the face mask except at an air inlet and an air outlet. Further, the air inlet can include first and second air inlets, and the air outlet can include first and second air outlets.

For yet another example, the face mask can also include an outer shell, an inner shell, and an intermediate shell located between the outer and inner shell. Further, the intermediate shell can define first ducting along a first air flow path in which first air is configured to flow to dissipate the heat generated by the first thermoelectric cooling device and define second ducting along a second air flow path in which second air is configured to flow to dissipate the heat generated by the second thermoelectric cooling device.

For another example, the cooling configured to be applied to skin under the first eye of the user and the cooling configured to be applied to skin under the second eye of the user can each be configured to be at a temperature in a range of 15° C. to 25° C.

In another implementation, a therapeutic device including a face mask configured to be worn on a face of a user. The face mask includes a first thermoelectric cooling device, a first air inlet, a first air outlet, a first fan, a second thermoelectric cooling device, a second air inlet, a second air outlet, and a second fan. The first thermoelectric cooling device is configured to be positioned under a first eye of the user, is configured to generate cooling configured to be applied to skin under the first eye of the user, and is configured to generate heat. The first air inlet is through which air external to the face mask is configured to enter a U-shaped first air flow path in the face mask. The first air outlet is through which the air is configured to exit the first air flow path to exit the face mask. The first fan is upstream of the first thermoelectric cooling device and is configured to cause the air to enter the face mask through the first air inlet and to flow along the first air flow path. The air flowing along the first air flow path is configured to dissipate the heat generated by the first thermoelectric cooling device. The second thermoelectric cooling device is configured to be positioned under a second eye of the user, is configured to generate cooling configured to be applied to skin under the second eye of the user, and is configured to generate heat. The second air inlet is through which air external to the face mask is configured to enter a U-shaped second air flow path in the face mask. The second air outlet is through which the air is configured to exit the second air flow path to exit the face mask. The second fan is upstream of the second thermoelectric cooling device and is configured to cause the air to enter the face mask through the second air inlet and to flow along the second air flow path. The air flowing along the second air flow path is configured to dissipate the heat generated by the second thermoelectric cooling device.

The therapeutic device can have any number of variations. For example, the first and second thermoelectric cooling devices can each be a Peltier device.

For another example, the first air inlet, the first air outlet, the second air inlet, and the second air outlet can each be located at a bottom of the face mask.

For yet another example, the face mask can also include a first heat sink located along the first air flow path downstream of the first fan, the face mask can also include a second heat sink located along the second air flow path downstream of the second fan, the air entering the first air inlet can be configured to flow to the first fan and then laterally inward to the first heat sink, and the air entering the second air inlet can be configured to flow to the second fan and then laterally inward to the second heat sink. Further, the air flowing laterally inward to the first heat sink can be in an opposite direction to the air flowing laterally inward to the second heat sink; and/or the face mask can also include first internal ducting and second internal ducting, the first internal ducting extending between the first air inlet and the first fan and extending from the first heat sink toward the first air outlet and the second internal ducting extending between the second air inlet and the second fan and extending from the second heat sink toward the second air outlet.

For another example, the face mask can be air tight around a perimeter of the face mask except at the first air inlet, the first air outlet, the second air inlet, and the second air outlet.

For yet another example, the face mask can also include an outer shell, an inner shell, and an intermediate shell located between the outer and inner shell, and the intermediate shell can define first ducting along the first air flow path and define second ducting along the second air flow path. Further, the first ducting can include first inflow ducting extending between the first air inlet and the first fan and first outflow ducting extending toward the first air outlet, and the second ducting can include second inflow ducting extending between the second air inlet and the second fan and second outflow ducting extending toward the second air outlet.

For still another example, the air flowing along the first air flow path can also be configured to exit the face mask through the second air outlet, and the air flowing along the second air flow path can also be configured to exit the face mask through the first air outlet.

For another example, the first and second air flow paths can together define a W shape.

For yet another example, the cooling configured to be applied to skin under the first eye of the user and the cooling configured to be applied to skin under the second eye of the user can each be configured to be at a temperature in a range of 15° C. to 25° C.

For another example, the therapeutic device can also include a control unit operably coupled to the face mask, and the control unit can be configured to receive an input from the user and to control, based on the input, the cooling configured to be applied to skin under the first eye of the user and the cooling configured to be applied to skin under the second eye of the user.

For still another example, the face mask can also include a plurality of LEDs configured to emit light toward and to skin on the face of the user with the user wearing the face mask. Further, the therapeutic device can also include a control unit operably coupled to the face mask, and the control unit can be configured to control the plurality of lights, the cooling configured to be applied to skin under the first eye of the user, and the cooling configured to be applied to skin under the second eye of the user; and/or the LEDs can each be configured to emit different wavelengths of light such as a first wavelength in a range of 450 nm to 495 nm (e.g., blue light), a second wavelength in a range of 620 nm to 740 nm (e.g., red light), and a third wavelength in a range of 800 nm to 2500 nm (e.g., infrared light).

In another aspect, a method is provided that in one implementation includes using any of the therapeutic devices described above.

### BRIEF DESCRIPTION OF DRAWINGS

This disclosure will be more fully understood from the following detailed description taken in conjunction with the accompanying drawings, in which:

FIG. 1 is a perspective view of one implementation of a face mask and a control unit connected to the face mask with a cable;

FIG. 2 is a perspective view of the face mask and a portion of the cable of FIG. 1;

FIG. 3 is another perspective view of the face mask and a portion of the cable of FIG. 1;

FIG. 4 is another perspective view of the face mask and a portion of the cable of FIG. 1 with removable pads removed from the face mask;

FIG. 5 is a perspective view of the face mask and a portion of the cable of FIG. 1 omitting an inner shell of the face mask;

FIG. 6 is a perspective view of the face mask and a portion of the cable of FIG. 1 omitting the inner shell of the face mask and with removable pads removed from the face mask;

FIG. 7 is another perspective view of the face mask and a portion of the cable of FIG. 1 omitting the inner shell of the face mask and with removable pads removed from the face mask;

FIG. 8 is a perspective view of a portion of the face mask of FIG. 1 omitting an outer shell of the face mask;

FIG. 9 is another perspective view of a portion of the face mask of FIG. 1 omitting the outer shell of the face mask;

FIG. 10 is yet another perspective view of a portion of the face mask of FIG. 1 omitting the outer shell of the face mask;

FIG. 11 is still another perspective view of a portion of the face mask of FIG. 1 omitting the outer shell of the face mask;

FIG. 12 is a perspective view of the control unit and a portion of the cable of FIG. 1;

FIG. 13 is another perspective view of the control unit and a portion of the cable of FIG. 1;

FIG. 14 is a side, partially cross-sectional view of the control unit and a portion of the cable of FIG. 1;

FIG. 15 is an exploded perspective view of a portion of the face mask of FIG. 1;

FIG. 16 is a perspective view of a portion of the face mask of FIG. 1 omitting non-removable pads of the face mask;

FIG. 17 is a perspective view of one implementation of a stand configured for use with the face mask of FIG. 1;

FIG. 18 is another perspective view of the control unit and a portion of the cable of FIG. 1;

FIG. 19 is a perspective view of another implementation of a face mask;

FIG. 20 is another perspective view of the face mask of FIG. 19;

FIG. 21 is a perspective view of the face mask of FIG. 19 omitting an inner shell of the face mask;

FIG. 22 is another perspective view of the face mask of FIG. 19 omitting the inner shell of the face mask and omitting the non-removable pads of the face mask;

FIG. 23 is a schematic view of one implementation of lights and thermoelectric cooling devices for a face mask;

FIG. 24 is a schematic view of another implementation of lights and thermoelectric cooling devices for a face mask;

FIG. 25 is a schematic view of yet another implementation of lights and thermoelectric cooling devices for a face mask; and

FIG. 26 is a schematic view of still another implementation of lights and thermoelectric cooling devices for a face mask.

### DETAILED DESCRIPTION

Certain embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape.

Various illustrative systems, devices, and methods for face masks are provided. In general, a face mask is configured to provide cooling therapy to a user wearing the face mask. In an exemplary implementation, the face mask includes a thermoelectric cooling device, such as a Peltier device, a thermoelectric cooler (TEC), or other thermoelectric cooling device. The thermoelectric cooling device is configured to generate heat through a thermoelectric effect where a heat flux is created at a junction of two different types of materials. The heat flux creates a cold area and a hot area. The cold area is configured to face toward the user's face to provide cool energy to the user's skin at the user's face. The hot area creates heat energy that the face mask is configured to dissipate to help prevent the heat from interfering adversely with the cooling provided to the user by the face mask. Dissipating the heat generated from the thermoelectric cooling device may help prevent the heat from interfering adversely with the cooling effect provided by the face mask.

The face mask includes a fan configured to blow air configured to dissipate the heat created by the thermoelectric cooling device. The face mask includes an air inlet through which air, e.g., ambient air, is configured to enter the face mask. With the fan on, the fan is configured to draw the air into the face mask through the air inlet. The face mask includes an air inflow path along which the air is configured to flow to the fan from the air inlet. The fan is configured to blow the air across the thermoelectric cooling device, e.g., across the hot side of the thermoelectric cooling device, and then along an air outflow path of the face mask to an air outlet through which the air is configured to exit the face mask. Air is thus configured to flow unidirectionally through the mask from the air inlet to the air outlet.

In an exemplary implementation, the face mask includes first and second thermoelectric cooling devices. The first thermoelectric cooling device is associated with a left eye opening of the face mask that is configured to align with a left eye of a user wearing the face mask. The second thermoelectric cooling device is associated with a right eye opening of the face mask that is configured to align with a right eye of the user wearing the face mask. The first and second thermoelectric cooling devices are configured to be located below the user's left and right eyes, respectively, to allow the cooling provided by the first and second thermoelectric cooling devices to be applied below the user's left and right eyes. Cooling applied below the user's left and right eyes is configured to depuff, soothe, and refresh the user's under-eye area by shrinking blood vessels under the eyes.

The methods, systems, and devices described herein apply to face covering devices configured to provide cooling therapy and light therapy to a user wearing the face covering device and to face covering devices that are configured to provide cooling therapy without being configured to provide light therapy. In an exemplary implementation in which a face covering device is configured to provide cooling therapy and light therapy, the face mask is configured to provide the cooling therapy and the light therapy simultaneously. Light cannot be felt by the user whereas the user will feel cooling, e.g., by feeling coolness at an area of the face adjacent the face mask's thermoelectric cooling device(s). Additionally, light therapy typically takes at least four weeks, and in some instances can take six to eight weeks or more, of daily mask use to provide a physical effect noticeable to the user, whereas cooling therapy typically provides a physical effect noticeable to the user after each session of receiving cooling therapy via the face mask. Cooling therapy may thus serve as an immediate physical signal to the user that the face mask is providing therapeutic effect while the user is wearing the mask and receiving cooling therapy and light therapy, e.g., because the user feels cooling, which may reassure that the face mask is working to provide treatment. Similarly, cooling therapy may serve as an immediate physical signal to the user that the face mask is providing therapeutic effect after each session of using the face mask, e.g., by noticing an effect in under-eye areas, whether or not light therapy is also being applied, which may improve user experience.

In an exemplary implementation in which the face covering device is configured to provide cooling therapy and light therapy to a user wearing the face covering device, the face covering device is also configured to, at a user's selection, provide cooling therapy and light therapy simultaneously or provide only one of cooling therapy and light therapy, thereby allowing for user-customized therapy. Over time a user's therapy needs may change, so the face mask being configured to provide user-customized therapy may extend the mask's useful life for a user. Additionally, different users may use the same mask, so the face mask being configured to provide user-customized therapy may allow each of the different users to select their own desired therapy, which may be cooling therapy only, light therapy only, or cooling therapy and light therapy.

In an exemplary implementation, the face mask's air inlet and air outlet are both located at a bottom of the face mask. Noise from air entering the air inlet and noise from air exiting the air outlet may thus be directed away from the user's ears, which are located by opposed left and right sides of the mask some distance above the bottom of the face mask. In some implementations, instead of being located at the bottom of the face mask, one or both of the air inlet and air outlet are located at a top of the face mask some distance above the user's ears, which is configured to direct air away from the user's ears similar to being located at the bottom of the face mask. In some implementations, instead of being located at the bottom or top of the face mask, one or both of the air inlet and air outlet are located at locations between a top of the face mask and a bottom of the face mask, such as by being substantially aligned with the user's eyes, by being substantially aligned with the user's cheeks, or at some other location. A particular face mask's geometry and construction may dictate where the air inlet and air outlet are located.

In some implementations, the face mask includes a noise attenuation system configured to attenuate the noise that is radiated into the environment, and thus heard by the user, due to use of the fan of the face mask. The noise attenuation system is configured to automatically attenuate noise, so the user does not need to take any particular action specific to reducing noise. Noise heard by the user may thus be automatically reduced whenever the face mask is on the user's face and the fan is running, thereby improving user experience. In implementations where the face mask includes more than one fan, the face mask can include multiple noise attenuations systems, one per fan, so noise generated by all the fans can be attenuated. Various exemplary implementations of noise attenuation systems are described further in, for example, U.S. patent application Ser. No. 18/411,644 entitled “Face Masks With Noise Attenuation” filed Jan. 12, 2024 and U.S. Pat. Pub. No. 2024/0257792 entitled “Acoustic Muffler For A Motorized Food Processing Device” published Aug. 1, 2024, which are hereby incorporated by reference in their entireties.

In an exemplary implementation, a noise attenuation system of a face mask includes an acoustic chamber upstream of the fan and a tortuous air path downstream of the fan. The acoustic chamber is located adjacent an air inlet through which air enters the face mask, e.g., under force provided by the fan, and is located along an air inflow path along which the air flows from the air inlet to the fan. The acoustic chamber has dimensions tuned to maximize noise reduction in a desired frequency band, e.g., a frequency band sensitive to human hearing. The air entering the air inlet of the face mask and flowing along the face mask's air inflow path causes noise that would be easily heard by the user without the acoustic chamber being present to attenuate the noise so as to reduce, if not eliminate, bothersome noise to the user causes by the air inflow.

The tortuous air path is located adjacent the air outlet through which air exits the face mask, e.g., under force provided by the fan, and defines at least a portion of an air outflow path along which the air flows from the fan to the air outlet. The tortuous air path has multiple twists and turns, which help attenuate noise of air flowing therethrough.

The noise attenuation system including the acoustic chamber upstream of the fan and the tortuous air path downstream of the fan is one example only. In some implementations, the noise attenuation system includes an acoustic chamber downstream of the fan and a tortuous air path upstream of the fan. In some implementations, the noise attenuation system includes a first acoustic chamber and a first tortuous air path upstream of the fan and a second acoustic chamber and a second tortuous air path downstream of the fan. In some implementations, the noise attenuation system includes a first acoustic chamber upstream of the fan and a second acoustic chamber and a tortuous air path downstream of the fan. In some implementations, the noise attenuation system includes a first tortuous air path upstream of the fan and an acoustic chamber and a second tortuous air path downstream of the fan. In some implementations, the noise attenuation system includes a first acoustic chamber and a tortuous air path upstream of the fan and a second acoustic chamber downstream of the fan. In some implementations, the noise attenuation system includes an acoustic chamber and a first tortuous air path upstream of the fan and a second tortuous air path downstream of the fan.

The noise attenuation system including at least one of an acoustic chamber and a tortuous air path along each of the inflow and outflow air paths is configured to help reduce noise caused by each of air inflow and air outflow. However, in some implementations, the noise attenuation system includes at least one of an acoustic chamber and a tortuous air path along only one of the inflow and outflow air paths, such as due to space constraints of a particular face mask, cost constraints of a particular face mask, or other reason. For example, the noise attenuation system can include an acoustic chamber upstream of the fan. For another example, the noise attenuation system can include an acoustic chamber downstream of the fan. For yet another example, the noise attenuation system can include a tortuous air path upstream of the fan. For still another example, the noise attenuation system can include a tortuous air path downstream of the fan. For another example, the noise attenuation system can include an acoustic chamber and a tortuous air path upstream of the fan. For yet another example, the noise attenuation system can include an acoustic chamber and a tortuous air path downstream of the fan.

In some implementations, the noise attenuation system includes, in addition to at least one of an acoustic chamber and a tortuous air path, a flared acoustic waveguide at the air inlet and/or a flared acoustic waveguide at the air outlet. The flared acoustic waveguide at the air inlet is located at an interface between the air inflow path and an external environment from which air flows into the face mask. The flared acoustic waveguide at the air inlet may thus reduce low-frequency sound radiation into the external environment, and thus to the user's ears, from the air inlet. The flared acoustic waveguide at the air outlet is located at an interface between the air outflow path and the external environment to which air exits from the face mask. The flared acoustic waveguide at the air outlet may thus reduce low-frequency sound radiation into the external environment, and thus to the user's ears, from the air outlet.

Various exemplary implementations of face masks are described further in, for example, U.S. patent application Ser. No. 18/411,644 entitled “Face Masks With Noise Attenuation” filed Jan. 12, 2024 and U.S. patent application Ser. No. 18/411,806 entitled “Light Emitting Face Masks” filed Jan. 12, 2024, which are hereby incorporated by reference in their entireties.

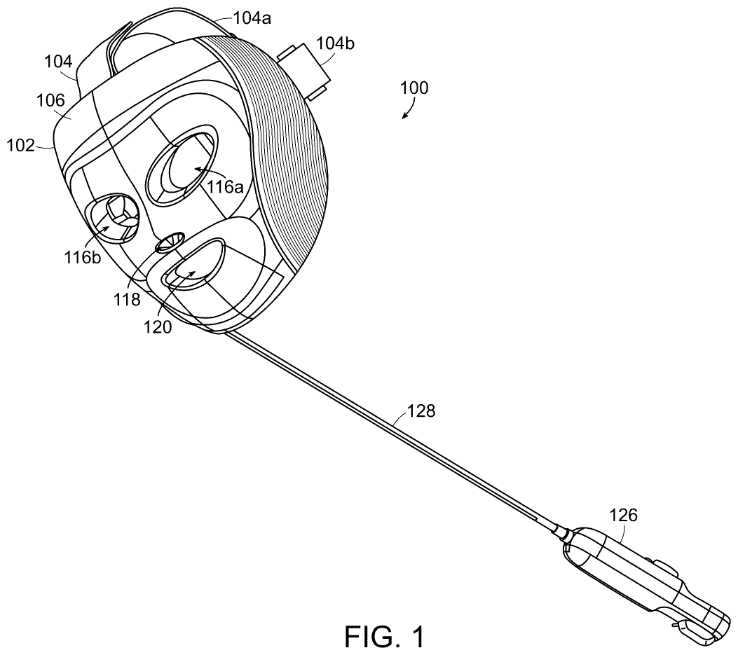

FIG. 1 illustrates one exemplary implementation of a face covering device (also referred to herein as a “face mask” or “mask”) 100 configured to provide cooling therapy to a user wearing the face covering device 100. The face covering device 100 in this illustrated implementation is also configured to provide light therapy to a user wearing the face covering device 100. However, as mentioned above, in some implementations the face covering device 100 is not configured to provide light therapy.

The mask 100 includes a base 102 and a support 104 attached to the base 102. The base 102 is configured to be worn over a user's face. The support 104 is configured to be worn on the user's head to support the mask 100, and thus the base 102, on the user's head.

The support 104 can have a variety of configurations. For example, the support 104 can include a cap configured to be worn on a user's head similar to a hat. For another example, as in this illustrated implementation as shown in FIGS. 1-4, the support 104 can include a strap assembly including a first strap 104a and a second strap 104b attached to the first strap 104a. The first strap 104a is an upper portion of the strap assembly and is configured to be worn over and extend front-back along a crown of a user's head. The second strap 104b is a lower portion of the strap assembly and is configured to be worn around and extend substantially horizontally along a partial circumference of the user's head. The second strap's extension may not be precisely horizontal but nevertheless be considered to be substantially horizontal, depending on a particular user's head and how a user positions the second strap 104b. In an exemplary implementation, the first strap 104a and the second strap 104b are made from a flexible material, e.g., a textile, a plastic, or a combination thereof, which may help the first strap 104a and the second strap 104b comfortably conform to a size and shape of particular user's head.

In some implementations, the strap assembly includes padding, e.g., foam, air pockets, or other padding, configured to be positioned between the user's head and each of the strap assembly's straps to provide increased user comfort.

The strap assembly includes an adjustment mechanism, such as a buckle, snaps, Velcro, or other adjustment mechanism, configured to allow manual user adjustment of the first strap 104a and the second strap 104b to help fit the mask 100 snugly and comfortably on the user. The mask 100 in this illustrated implementation includes an adjustment mechanism in the form of Velcro (obscured in the figures) for adjustment of the first strap 104a and in the form of first and second sliders 104c, 104d for adjustment of left and right sides, respectively, of the second strap 104b. In other implementations, the strap assembly is self-adjusting, such as by straps of the strap assembly being elastic members similar to an elastic headband.

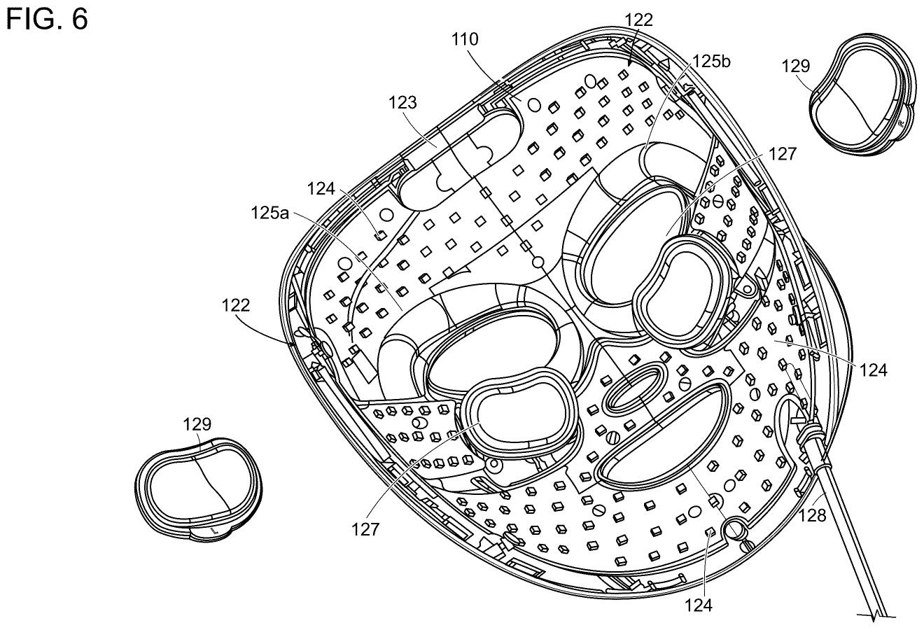

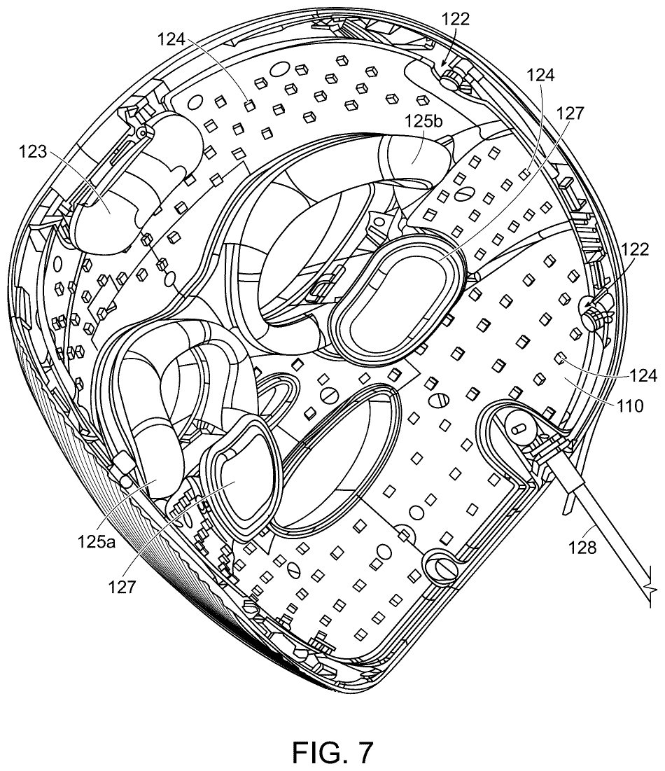

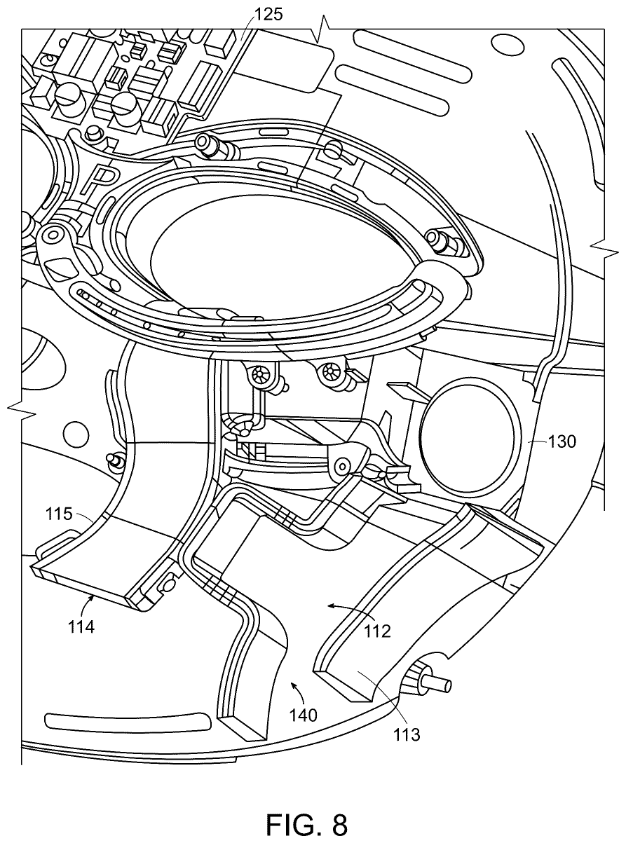

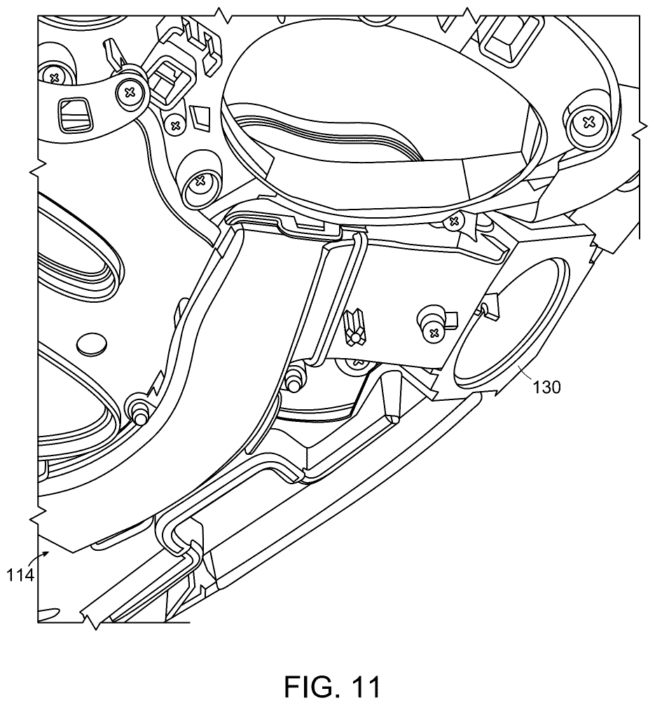

As shown in FIGS. 1-7, the base 102 includes an outer shell 106, an inner shell 108, and an intermediate shell 110 located between the outer and inner shells 106, 108. In an exemplary implementation, the base 102 is made from a rigid material, e.g., a plastic, a metal, or a combination thereof, which may help prevent the mask 100 from bending, deflecting, twisting, or otherwise breaking and/or may help prevent first and second air inflow paths 112 (see FIGS. 8-10) of the mask 100 and first and second air outflow paths 114 (see FIGS. 8 and 11) of the mask 100, which are defined in the mask 100 by ducting formed between the outer and inner shells 106, 108, from deforming, twisting, or otherwise becoming at least partially obstructed so as to impede air flow. The second air inflow path and the second outflow path in the mask 100 on a right side of the mask 100 are obscured in the figures but are similar to the first air inflow path 112 and first air outflow path 114 on a left side of the mask 100.

The mask 100 includes a plurality of openings 116a, 116b, 118, 120 formed therein that each corresponds to a face feature and is configured to align at least partially with the face feature when the mask 100 is worn by a user. In this way, with a user wearing the mask 100, each of the plurality of openings 116a, 116b, 118, 120 will align at least partially with a feature of the user's face. Each of the plurality of openings 116a, 116b, 118, 120 is formed in the base 102 and is formed through all of the outer, inner, and intermediate shells 106, 108, 110. The plurality of openings include a first eye opening 116a configured to align at least partially with a right eye of a user wearing the mask 100, a second eye opening 116b configured to align at least partially with a left eye of the user wearing the mask 100, a nose opening 118 configured to align at least partially with a nose of the user wearing the mask 100, and a mouth opening 120 configured to align at least partially with a mouth of the user wearing the mask 100. The first and second eye openings 116a, 116b are configured to allow the user to see while wearing the mask 100 without the mask 100 preventing the user from being able to see anything except an inside surface of the mask 100, e.g., an inside surface of the inner shell 108. The nose opening 118 is configured to allow the user to easily use their nose, e.g., for breathing, etc., while the user is wearing the mask 100. The mouth opening 120 is configured to allow the user to easily use their mouth, e.g., for breathing, drinking, eating, etc., while the user is wearing the mask 100.

All of the plurality of openings 116a, 116b, 118, 120 in this illustrated implementation are unobstructed openings. In other implementations, one or more of the plurality of openings 116a, 116b, 118, 120 can be at least partially obstructed, such as with mesh, a transparent polymer plate, or other obstruction element.

The base 102 in this illustrated implementation is configured to cover substantially all of a user's face with the user wearing the mask 100. The base 102 may not entirely cover a particular user's face depending on a size and shape of the particular user's face, but the base 102 has a size configured to cover faces of most potential users of the mask 100. The base 102 thus includes openings 116a, 116b, 118, 120 for all of the user's eyes, nose, and mouth. In some implementations, the base 102 is configured to partially cover a user's face, such as only cover an upper half of a user's face, only cover a lower half of a user's face, cover a user's face except for left and right cheeks, or other partial coverage configuration. In such implementations, the base 102 may not have at least one of the eye, nose, and mouth openings 116a, 116b, 118, 120 depending on where the mask 100 is intended to be placed over a user's face. For example, whether or not the mask 100 is configured to provide light therapy, the mask 100 may not extend down to a user's mouth and thus not have the mouth opening 120 since, as discussed further below, cooling therapy is only applied under the user's eyes.

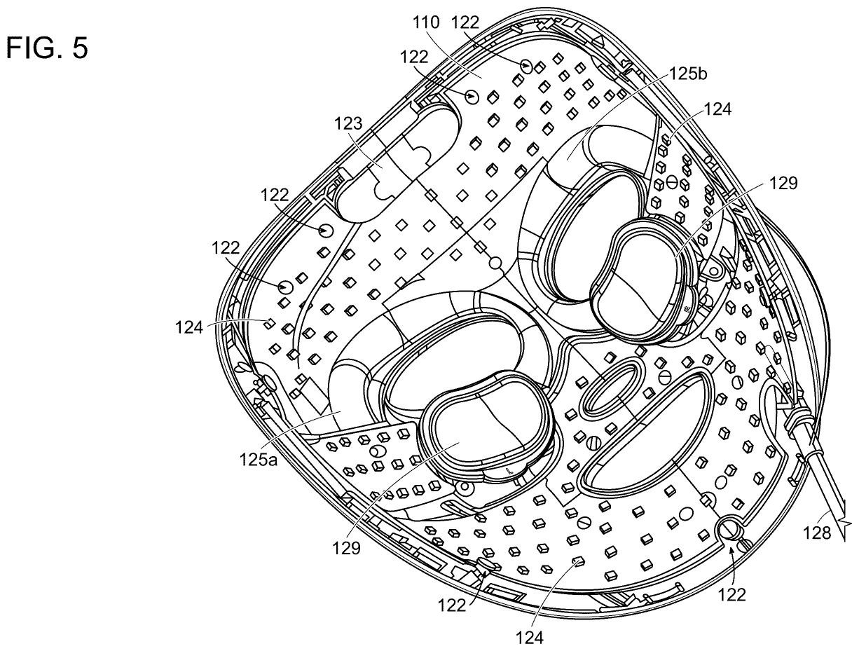

The outer shell 106 defines an exterior surface of the mask 100 that faces away from a user's face with the user wearing the mask 100. The inner shell 108 defines an interior surface of the mask 100 that faces toward a user's face with the user wearing the mask 100. The intermediate shell 110 is sandwiched between the outer and inner shells 106, 108. The mask 100 includes at least one interior connection point at which the outer, inner, and intermediate shells 106, 108, 110 are configured to be securely attached together, such as by using pins, adhesive, welding, etc. In this illustrated implementation, the outer and inner shells 106, 108 include protrusions that attach together through cut-outs 122 (see FIGS. 5-7) formed in the intermediate shell 110. The illustrated mask 100 includes nine interior connection points, but another number of interior connection points can be used.

A light assembly is located on the intermediate shell 110. The light assembly is configured to apply light therapy to a user wearing the mask 100. The light assembly includes a plurality of lights 124 spaced apart from one another in a pattern, e.g., a grid pattern, a random pattern, or other pattern, on the intermediate shell 110.

The light 124 are configured to be selectively turned on by a user, to provide the light therapy, and off by the user, to not be providing the light therapy. The lights 124 are light emitting diodes (LEDs) in this illustrated implementation. A number of the lights 124 can be, for example, in a range between ten and five hundred; in a range between two hundred and five hundred; in a range between three hundred and five hundred; in a range between four hundred and five hundred; in a range between ten and two hundred; in a range between fifty diodes and one hundred fifty; in a range between sixty and one hundred; in a range between seventy-five and eighty-five; fifty; seventy-five; eighty; eighty-five; two hundred, two hundred forty, four hundred thirty, four hundred fifty, four hundred eighty, five hundred, or other number.

Each of the lights 124 is configured to emit light at at least one predetermined wavelength configured to facilitate various light therapies, such as one or more of an anti-aging treatment and an anti-breakout treatment. The predetermined wavelength can be, for example, a wavelength in a range between about 300 nm and about 1000 nm, or, for another example, in a range of about 450 nm to 2500 nm. The light emitted by the plurality of lights 124 is configured to reach one or more layers of skin, e.g., an epidermis, a dermis, and/or a hypodermis, of a user wearing the mask 100. The layer(s) of skin reached by the light corresponds to the wavelength. For example, a longer wavelength of light is configured to reach a deeper layer of skin than a shorter wavelength of light. In some implementations, the wavelength emitted by the lights 124 is configured to be adjustable, such as by one or more of a user manually selecting the wavelength and a wireless computing device configured to wirelessly communicate with a controller of the face covering device 100 to allow a user to manually select the wavelength.

In an exemplary implementation, each of the lights 124 is configured to emit red light, blue light, and infrared (IR) light. By being configured to emit light at different wavelengths (red wavelength, blue wavelength, and IR wavelength), energy from the lights 124 is configured to provide therapeutic benefit at different skin depths. IR light has a higher wavelength (e.g., in a range of 800 nm to 2500 nm) than blue light wavelength (e.g., in a range of 450 nm to 495 nm) and red light wavelength (e.g., in a range of 620 nm to 740 nm) so is configured to penetrate deeper into the skin than blue light and red light. The IR light energy emitted by the lights 124 is configured to be absorbed by skin, e.g., cells of skin, to provide one or more therapeutic benefits such as reducing deep wrinkles, preventing future fine lines, increasing collagen, and/or expanding blood vessels to allow for better blood flow. Red light has a higher wavelength than blue light so is configured to penetrate deeper into the skin than blue light. The red light energy emitted by the lights 124 is configured to be absorbed by skin, e.g., cells of skin, to provide one or more therapeutic benefits such as producing collagen to aid in anti-aging (e.g., smoothing wrinkles, increase skin firmness, reduce skin sagging, reducing fine lines, etc.). Blue light has a lower wavelength than red light and IR light and is configured to affect the skin's surface. The blue light energy emitted by the lights 124 is configured to be absorbed by skin, e.g., cells of skin, to provide one or more therapeutic benefits such as balancing skin texture and treating breakouts.

In some implementations, each of the lights 124 is configured to emit only red light. In some implementations, each of the lights 124 is configured to emit only blue light. In some implementations, each of the lights 124 is configured to emit only IR light. In some implementations, each of the lights 124 is configured to emit only red light and blue light. In some implementations, each of the lights 124 is configured to emit only blue light and IR light. In some implementations, each of the lights 124 is configured to emit only red light and IR light.

Various exemplary implementations of light assemblies for a face mask are described further in, for example, U.S. patent application Ser. No. 18/901,465 entitled “Therapeutic Face Mask Modes Of Operation” filed on Sep. 30, 2024 and U.S. patent application Ser. No. 18/411,806 entitled “Light Emitting Face Masks” filed Jan. 12, 2024, which are hereby incorporated by reference in their entireties.

The lights 124 are configured to provide therapeutic light therapy. In some implementations, the mask 100 includes at least one light configured to emit light for a non-therapeutic purpose. For example, the mask 100 can include at least one light configured to illuminate when the mask 100 is powered on to assure a user that the mask 100 has been powered on. For another example, the mask 100 can include at least one light configured to illuminate during the mask's application of cooling therapy. Cooling therapy applied to a user's skin does not have a visual element detectable by a user, unlike light therapy which will typically be perceptible visibly to a user wearing the mask 100 since light is being directed toward the user's face, so the light(s) may assure the user that the cooling therapy is being provided. In an exemplary implementation, at least one light (e.g., a single light, a pair of lights, a row of five lights, a row of ten lights, etc.) is located under the left eye opening 116a for visualization by a left eye of a user wearing the mask 100 and at least one light is located under the right eye opening 116b for visualization by a right eye of the user wearing the mask 100.

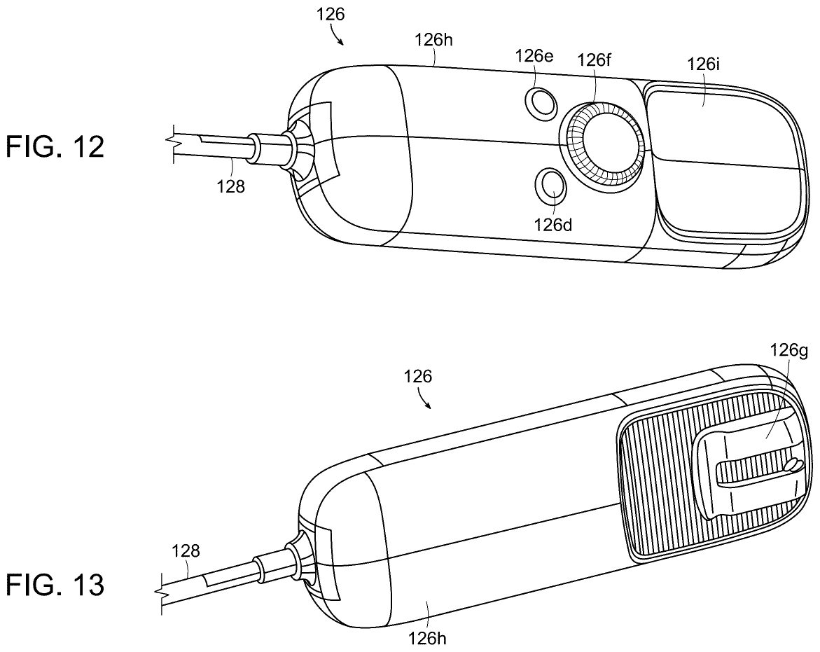

As shown in FIG. 1, a control unit 126 is connected to the mask 100. FIGS. 12-14 also show the control unit 126. The control unit 126 is configured to allow a user to control various functions of the mask 100. The control in this illustrated implementation includes control of the light therapy (e.g., turning the lights 124 on/off and adjusting light wavelength), control of the cooling therapy (e.g., turning cooling therapy on/off and adjusting cooling strength), and power control (on/off).

The control unit 126 is connected to the mask 100 with a cable 128. FIG. 1 shows the cable 128 connected at a first end of the cable 128 to the control unit 126 and at a second end of the cable 128 to the mask 100. The cable 128 extends from a lower portion of the mask 100 in this illustrated implementation, which may help the cable 128 be as unobtrusive as possible while a user is wearing the mask 100. The cable 128 is fixedly attached to the mask 100 and the control unit 126, which may help prevent loss of one of the mask 100 and the control unit 126 and/or may help ensure proper electrical connection between the mask 100 and the control unit 126. In other implementations, the cable 128 can be detachable from one or both of the mask 100 and the control unit 126, which may facilitate cleaning of the mask 100 (e.g., if the cable 128 is detachable from the mask 100), may allow for the mask 100 and/or control unit 126 to be replaced due to damage or other reason (e.g., if the cable 128 is detachable from the damaged or otherwise undesirable mask 100 and/or control unit 126), and/or may allow for an upgraded control unit to be used with the mask 100 (e.g., if the cable 128 is detachable from the mask 100).

The control unit 126 in this illustrated implementation is wired and is a dedicated control unit for the mask 100 and thus cannot control other masks 100 or other devices. In other implementations, the control unit 126 is a dedicated control unit for the mask 100 but is a wireless remote control configured to connect wirelessly to the mask 100, e.g., via Bluetooth or other wireless communication protocol. The control unit 126 being configured to connect wirelessly to the mask 100 may improve user experience by not requiring a user wearing the mask 100 to hold or clip the control unit 126. In still other implementations, the control unit 126 is not a dedicated control unit for the mask 100 and can control other masks and/or other devices. Examples of non-dedicated control units include a mobile phone, a mobile tablet, and other computing devices configured to wirelessly communicate with the mask 100.

The control unit 126 includes a power source 126a, a printed circuit board (PCB) 126b including electronic components (e.g., a controller, a memory, a bus, etc.), a display 126c, a plurality of controls 126d, 126e, 126f, a clip 126g, and an outer housing 126h. In this illustrated implementation, a protective lens 126i is disposed over the display 126c to help protect the display 126c from scratches or other damage.

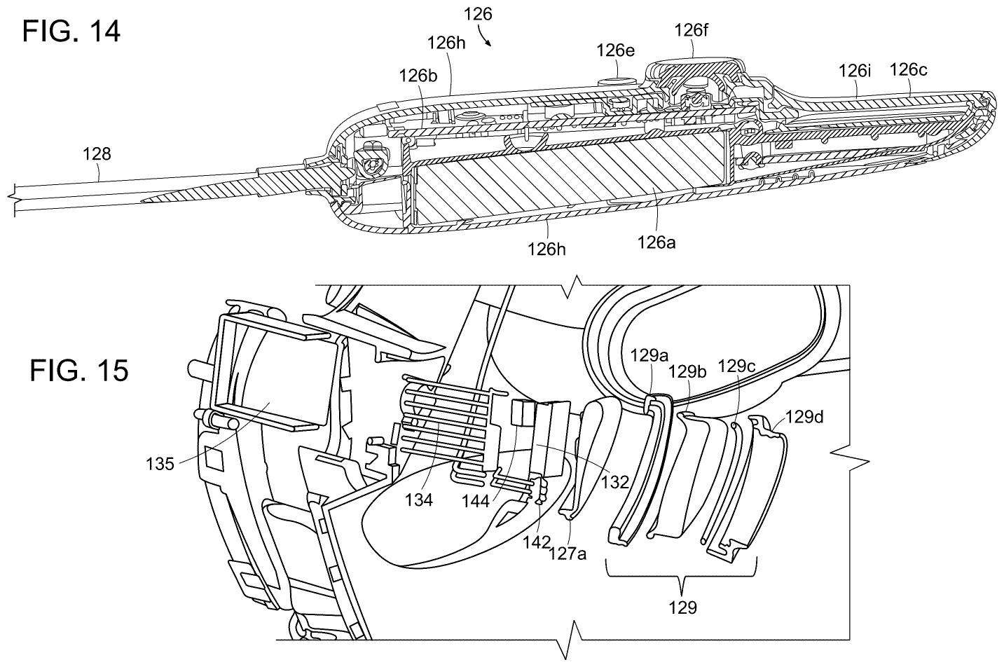

The power source 126a is configured to provide power to the PCB 126b of the control unit 126, to the lights 124 of the mask 100, and to a cooling system of the mask 100, which is discussed further below. The power source 126a includes a pair of rechargeable batteries in this illustrated implementation (only one of the batteries is visible in the view of FIG. 14), but another number of batteries or another power source can be used. The control unit 126 including the power source 126a instead of the mask 100 may help reduce a weight and/or bulkiness of the mask 100, which may provide for a better user experience. In other implementations, the mask 100 includes a power source instead of or in addition to the control unit 126.

The power source 126a in this illustrated implementation is attached to the PCB 126b via soldering, although another attachment mechanism can be used. Soldering the power source 126a to the PCB 126b may help reduce a profile of the control unit 126 because the power source 126a can be closer to the PCB 126b and the outer housing 126h, which may allow for a smaller control unit. A smaller control unit 126 may be lighter and easier for a user to hold or clip. A smaller control unit may allow for the control unit 126 to be less obtrusive during use of the mask 100, e.g., by more easily fitting in a user's pocket, by extending less from a user's body when clipped using the clip 126g, etc.

The plurality of controls 126d, 126e, 126f are configured to be actuated by a user to provide inputs to the control unit 126 for controlling various functionalities of the mask 100. The plurality of controls 126d, 126e, 126f in this illustrated implementation includes first, second, and third controls 126d, 126e, 126f. In other implementations, the control unit 126 includes a single control or a different plural number of controls, e.g., two, four, five, etc.

The first control 126d is a cooling button configured to be actuated by a user, e.g., pressed down, to turn cooling therapy on and off. In other implementations, another type of cooling control can be used, such as a toggle switch, a lever, or other control.

The second control 126e is a back button configured to be actuated by a user, e.g., pressed down, to move back to an immediately previous screen shown on the display 126c. In other implementations, another type of back control can be used, such as a toggle switch, a knob, or other control.

The third control 126f is a knob configured to be actuated by a user in a first motion, e.g., rotated, to scroll through options shown on the display 126c, to be actuated by a user in a second motion, e.g., pushed down for less than a predetermined amount of time, to select an option shown on the display 126c, and to be actuated by a user in a third motion, e.g., pushed down for at least the predetermined amount of time, to turn the control unit 126 on or off. In other implementations, one or more other type of controls can be used for scrolling through options, for selecting an option, and for power on/off, such as a toggle switch, lever, or other control.

The plurality of controls 126d, 126e, 126f are each operatively coupled to the PCB 126b of the control unit 126. In response to one of the plurality of controls 126d, 126e, 126f being actuated, the PCB 126b of the control unit 126 is configured to control the control unit 126 to cause the selected action to occur, e.g., power the control unit 126 on or off, show different information on the display 126c, cause cooling therapy to begin, cause light therapy to begin, etc.

The display 126c can be any of a variety of types of displays, such as a liquid crystal display (LCD) display or other type of display. In some implementations, instead of or in addition to the control unit 126 including the control(s) configured to be actuated by a user to provide inputs to the control unit 126, the display 126c is a touchscreen configured to receive input from a user.

The display 126c is configured to show information to the user regarding the mask 100. The information shown on the display 126c can include, for example, a power status (on/off), a battery charge level indication, a selected light therapy mode (also referred to herein as “light modes”), a selected cooling therapy mode (also referred to herein as “cooling modes”), an error status (e.g., an error code, a message explaining an error encountered, etc.), stored settings, stored historical use data (e.g., number of times and/or dates light therapy has been delivered from the mask 100, number of times and/or dates cooling therapy has been delivered from the mask 100, etc.), current date, current time, etc.

Various exemplary implementations of modes for a face mask and information regarding a face mask that may be shown to a user are described further in, for example, U.S. patent application Ser. No. 18/901,465 entitled “Therapeutic Face Mask Modes Of Operation” filed on Sep. 30, 2024, which is hereby incorporated by reference in its entirety.

The clip 126g of the control unit 126 is configured to allow the control unit 126 to be clipped to an item at a convenient location, e.g., a pocket, a collar, a shirt cuff, a waistband, a blanket, etc., so a user wearing the mask 100 does not have to hold the control unit 126 by hand while the mask 100 is providing cooling therapy and/or light therapy. Cooling therapy and light therapy each typically last at least a few minutes (e.g., in a range of about four minutes to about fifteen minutes), so user experience may be improved by the user not having to hold the control unit 126 by hand throughout the light therapy and/or cooling therapy. A person skilled in the art will appreciate that a value may not be precisely at a value but nevertheless be considered to be about that value for any of a variety of reasons, such as sensitivity of measurement equipment or manufacturing tolerances.

The clip 126g is located on a back side of the control unit 126 that is opposite to a front side of the control unit 126 where the plurality of controls 126d, 126e, 126f and the display 126c are located. The plurality of controls 126d, 126e, 126f are thus configured to be easily accessible and the display 126c is configured to be easily visible when the control unit 126 is clipped to clothing or other item with the clip 126g. The clip 154g can, however, be located elsewhere.

The illustrated control unit 126 does not include any lights, but in other implementations the control unit 126 can include one or more lights configured to provide information to a user. For example, a light being on can indicate that power is on and the light being off can indicate that power is off. For another example, a light being on can indicate that cooling therapy is being provided and the light being off can indicate that cooling therapy is not being provided. For yet another example, a light being on can indicate that light therapy is being provided and the light being off can indicate that light therapy is not being provided.

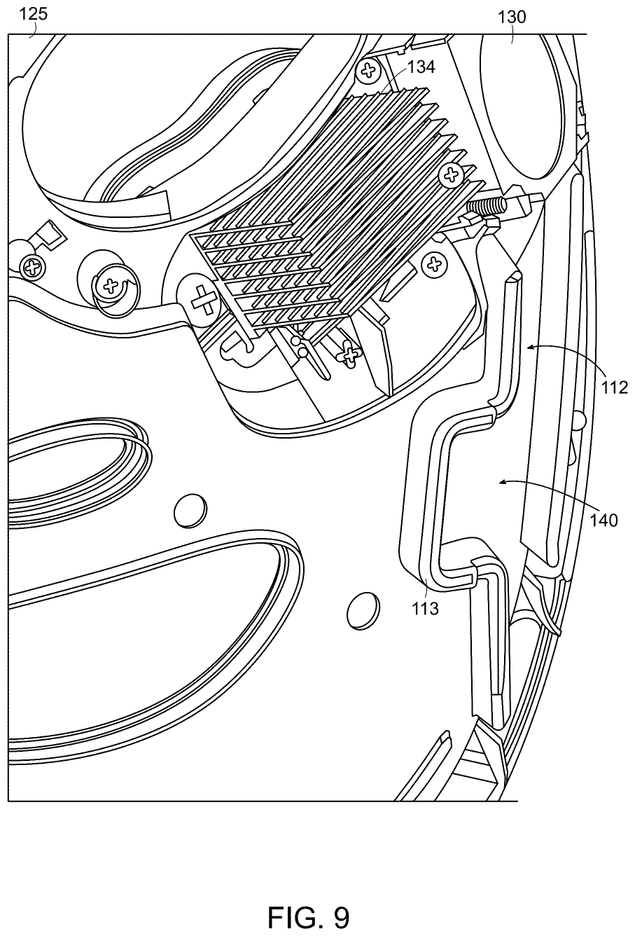

The mask 100 includes a PCB 125 (see FIGS. 8 and 9) including electronic components (e.g., a controller, a memory, a bus, etc.) configured to be in operable communication with the control unit 126. In this illustrated implementation the PCB 125 of the mask 100 is located in an upper portion of the mask 100 in a forehead area of the mask 100. Because of the relatively large amount of surface area in the forehead area of the mask 100, the PCB 125 being located in the upper portion of the mask 100 may allow for a larger, and thus more powerful, controller and other electronic components. In another implementation, the PCB 125 is located in a lower portion of the mask 100 in a chin area of the mask 100. The PCB 125 being in the lower portion of the mask 100 may ease manufacturing of the mask 100 for operable coupling of the control unit 126 and the PCB 125 such as if the cable 128 extends from the lower portion of the mask 100.

In this illustrated implementation, each of the control unit 126 and the mask 100 includes a PCB 126b, 125. In some implementations, only one of the control unit 126 and the mask 100 includes a PCB, which may reduce cost and/or ease manufacturing.

As mentioned above, the mask 100 includes a cooling system configured to provide cooling therapy to a user wearing the mask 100. The mask 100 is configured to provide cooling therapy via first and second pads configured to be positioned against skin under left and right eyes, respectively, of a user wearing the mask 100. Cooling applied below the user's left and right eyes is configured to depuff, soothe, and refresh the user's under-eye area.

The mask 100 is configured to be one-size-fits-all. However, because different people have different face shapes and contours, the first and second pads of the mask 100 may feel more comfortable and/or have better contact with skin for some users than for other users. Better contact with skin more effectively provides the cooling effect to the user. The mask 100 includes a plurality of sets of pads configured to be selectable by a user. The user may thus use the mask 100 with the set of pads that feels most comfortable and/or has the best skin contact. Additionally, by being usable with a selected set of pads, the same mask 100 may be more effectively used by different users as compared to a mask that does not include selectable pads.

In this illustrated implementation, the mask 100 is configured to be used with first and second sets of pads 127, 129 that each include two pads, one associated with each eye. In other implementations, the mask 100 can be configured to be used with only a single set of pads or with another plural number of sets of pads, e.g., three, four, etc.

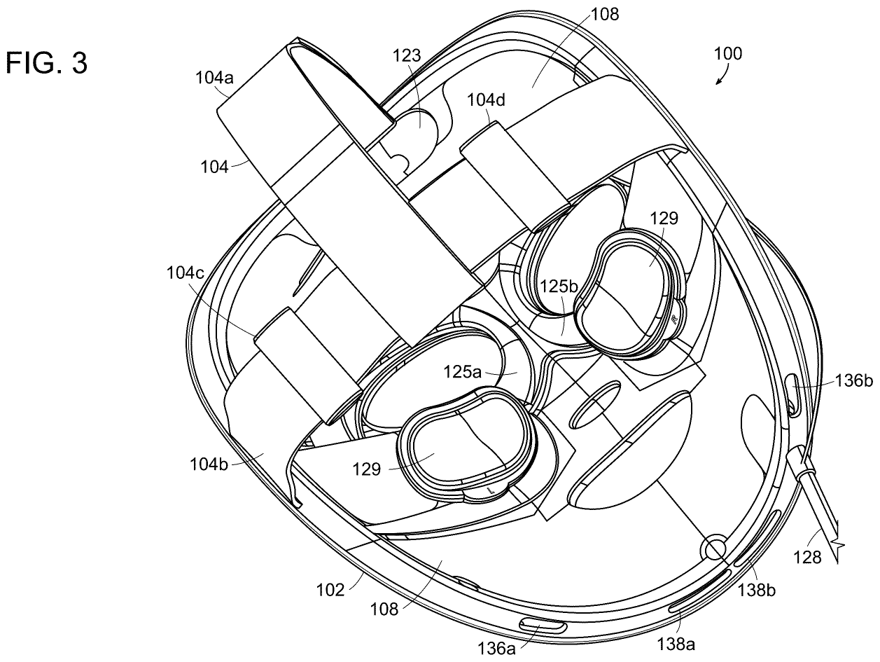

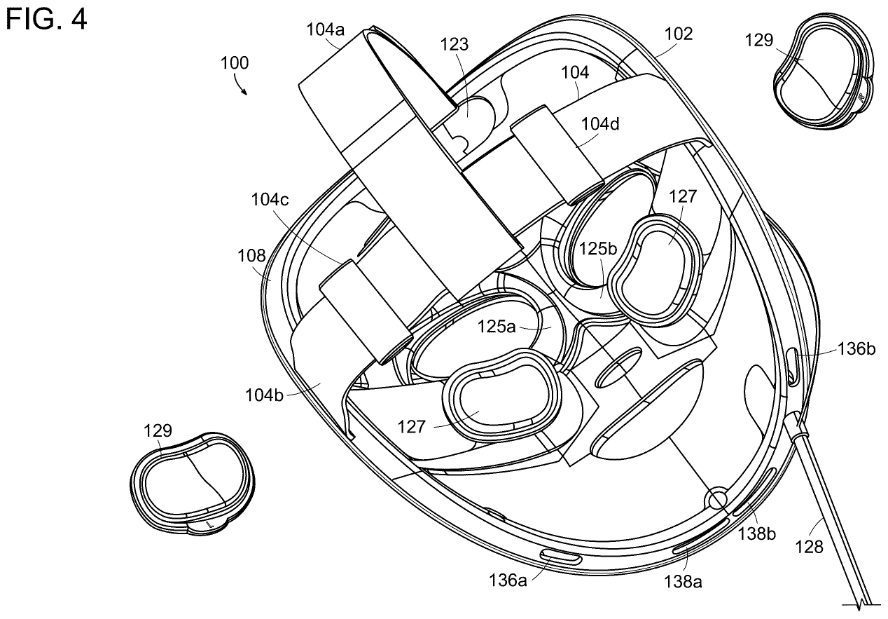

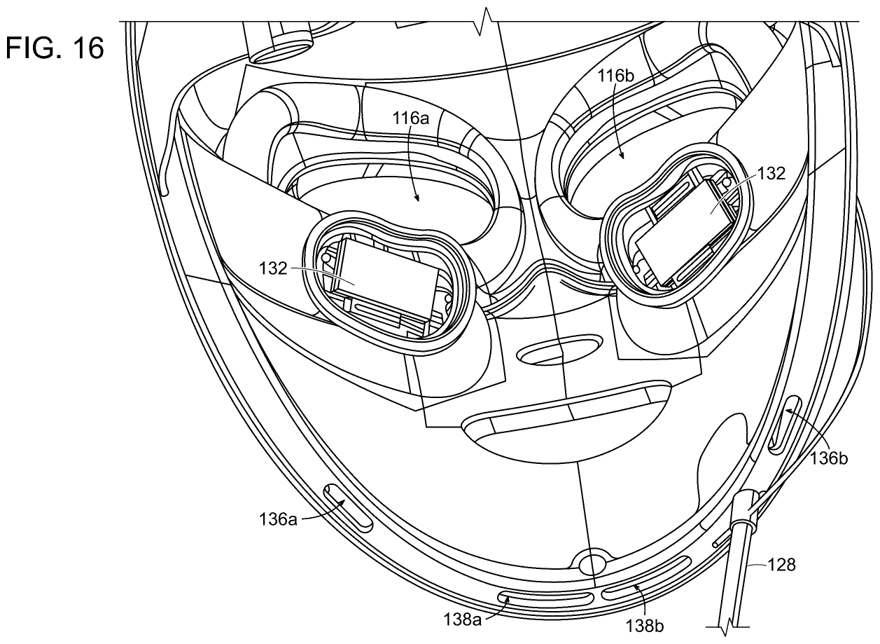

FIGS. 4, 6, and 7 show the mask 100 with the first set of pads 127 attached to the mask 100 without the second set of pads 129 attached to the mask 100. FIGS. 3 and 5 show the mask 100 with the second set of pads 129 attached to the mask 100 and the first set of pads 127 underlying the second set of pads 129, as discussed further below. FIG. 16 shows the mask 100 without either the first set of pads 127 or the second set of pads 129 attached to the mask 100 for purposes of illustration, since the first set of pads 127 is non-removably attached to the mask 100 in this illustrated implementation.

The first set of pads 127 is non-removably attached to the mask 100. By having a set of non-removable pads 127, the mask 100 prevents a user from using the mask 100 without pads, which may be uncomfortable and/or provide too much cooling of skin that could cause skin damage. The first set of pads 127 thus defines default pads of the mask 100.

The second set of pads 129 is configured to be removably attached to the mask 100. The user may thus select whether to use the first set of pads 127 against the user's face, e.g., the second set of pads 129 is not attached to the mask 100, or to use the second set of pads 129 against the user's face, e.g., the second set of pads 129 is attached to the mask 100.

The second set of pads 129 is configured to be removably attached to the mask 100 by clipping to the first set of pads 127. As shown in FIG. 15, which shows one of the pads of the second set of pads 129 as a representative example of both pads of the second set of pads 129, the pad 129 include a lower clip 129a, a coolsink 129b, a compressive layer 129c, and an upper clip 129d. The lower and upper clips 129a, 129d are attached together to hold together the coolsink 129b and the compressive layer 129c.

FIG. 15 also shows one of the pads of the first set of pads 127 as a representative example of both pads of the first set of pads 127. As shown in FIG. 15, the pad 127 includes a coolsink. Thus, with the second set of pads 129 removably attached to the mask 100, two coolsinks are used together.

The second set of pads 129 is configured to overlie the first set of pads 127 with the second set of pads 129 removably attached to the mask 100. The second set of pads 129 is thus configured to extend farther beyond the mask 100 in a direction toward a user's face than the first set of pads 127 and thus may feel more comfortable to some users than the first set of pads 127.

Each of the pads of the first and second sets of pads 127, 129 have different curvature based on whether the pad is for positioning under a left eye or under a right eye, as a person's face has different curvatures under the eyes. The second set of pads 129 includes left and right identifiers to indicate to a user where the pad 129 should be attached to the mask 100, e.g., under the left eye opening 116a or under the right eye opening 116b. The identifiers in this illustrated implementation are the letters “L” (for left) and “R” (for right), but other identifiers can be used additionally or alternatively, such as the words “left” and “right,” the left pad 129 being a first color that matches a color of the left pad of the first set of pads 127 and the right pad 129 being a second color that is different than the first color that matches a color of the right pad of the first set of pads 127, etc. The identifiers are on the upper clip 129d in this illustrated implementation but can be located elsewhere.

In some implementations, each of the first and second sets of pads 127, 129 is configured to removably attach to the mask 100, which may facilitate cleaning of the pads 127, 129.

In some implementations, the mask 100 does not include any removable sets of pads. Instead, the mask 100 includes only a non-removable set of pads, which may help prevent loss of pads and/or effectively prevent a user from using the mask 100 without pads.

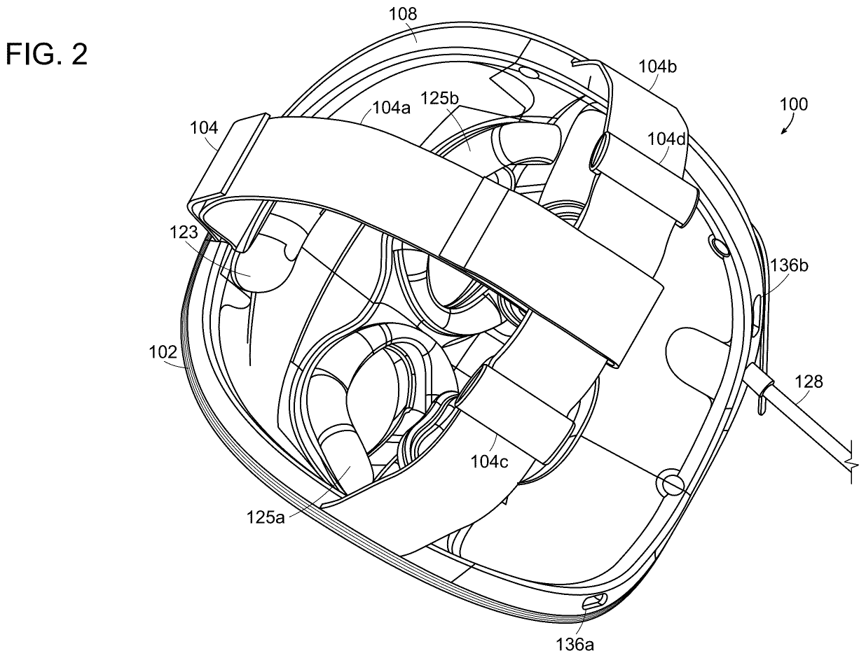

As shown in FIGS. 2-7, the mask 100 in this illustrated implementation includes a forehead pad 123 and first and second eye shields 125a, 125b. In other implementations, the forehead pad 123 and/or the first and second eye shields 125a, 125b are omitted.

The forehead pad 123 is configured to provide padding between the rigid base 102 and a forehead of a user wearing the mask 100 to improve user comfort and to help space the lights 124 a distance from the user's face. The forehead pad 123 is pill-shaped in this illustrated implementation but can have other shapes. The forehead pad 123 is a single pad, e.g., a single foam pad, in this illustrated implementation but can include multiple pads.

The first and second eye shields 125a, 125b are configured to provide padding between the rigid base 102 and areas around eyes of a user wearing the mask 100 to improve user comfort, to help space the lights 124 a distance from the user's face, and, when the mask 100 is providing light therapy, to help block light emitted from the lights 124 from distractingly shining into the user's eyes. The first and second eye shields 125a, 125b are each a single shield, e.g., a single silicone or other flexible member, in this illustrated implementation but can include multiple shields.

The first eye shield 125a extends around a portion of the first eye opening 116a and is thus associated with a left eye of a user wearing the mask 100. The first eye shield 125a extends around a partial perimeter of the first eye opening 116a to avoid interfering with cooling provided via the first cooling system, which is described further below. The second eye shield 125b extends around a portion of the second eye opening 116b and is thus associated with a right eye of a user wearing the mask 100. The second eye shield 125b extends around a partial perimeter of the second eye opening 116b to avoid interfering with cooling provided via the first cooling system, which is described further below.

The mask's cooling system can have a variety of configurations. As in this illustrated implementation, as shown in FIGS. 8-11, 15, and 16, the cooling system can include a fan 130, a thermoelectric cooling device 132, and a heat sink 134. In this illustrated implementation, the first heat sink 134 is made from aluminum but can have other configurations. The thermoelectric cooling device 132 is a Peltier device in this illustrated implementation but can be another type of thermoelectric cooling device such as a TEC or other thermoelectric cooling device. In this illustrated implementation, the Peltier device includes two ceramic plates with diodes located between the ceramic plates.

The mask 100 can include a single cooling system or, as in this illustrated implementation, can include a plurality of cooling systems. The illustrated mask 100 includes two cooling systems, a first cooling system and a second cooling system. The first cooling system is associated with a left side of the mask 100 and thus with a left side of the user's face and the user's left eye when the mask 100 is on the user's face. The second cooling system is associated with a right side of the mask 100 and thus with a right side of a user's face and the user's right eye when the mask 100 is on the user's face. The mask 100 therefore includes first and second fans 130, first and second thermoelectric cooling devices 132, and first and second heat sinks 134. The second fan and the second heat sink are obscured in the figures. The first and second cooling systems are configured and used similarly so are not each particularly described, with features described for the first cooling system similarly applying to the second cooling system.

The first thermoelectric cooling device 132 is configured to generate heat through a thermoelectric effect where a heat flux is created at a junction of two different types of materials, as will be appreciated by those skilled in the art. The heat flux creates a cold area and a hot area. The cold area is configured to face toward the user's face when the mask 100 is on the user face to provide cool energy to the user's skin, e.g., via contact of either the first set of pads 127 or the second set of pads 129 with the user's skin. The first pad 127 is a coolsink configured to transfer cooling from the first thermoelectric cooling device 132 to the user's skin (either directly or through the second pad 129, which is also a coolsink), and the other pad 127 of the first set of pads 127 is a coolsink configured to transfer cooling from the second thermoelectric cooling device to the user's skin (either directly or through the second pad 129, which is also a coolsink). The first and second sets of pads 127, 129 are metallic to facilitate the cold transfer, although another material is possible. Metal may more effectively transfer cold than other materials.

The hot area is configured to face away from the user's face, when the mask 100 is on the user face, to urge heat away from the user's skin. The first heat sink 134 faces the hot area of the first thermoelectric cooling device 132 to help the first heat sink 134 receive heat energy from the first thermoelectric cooling device 132, e.g., from the hot area thereof, to help prevent the heat from being applied to the user's face or interfering with the cooling effect provided to the user's skin via the first thermoelectric cooling device 132.

The first heat sink 134 is located downstream of the first fan 130. The first fan 130 is configured to blow air toward the first heat sink 134. The first fan 130 is thus configured to help dissipate the heat created by the first thermoelectric cooling device 132. Heat is therefore urged away from the first heat sink 134 and into a first cooling duct 135 that leads to the first air outflow path 114 to allow the air to exit out of the mask 100. The first fan 130 is operably coupled with the mask's controller to allow the mask's controller to control the first fan 130, e.g., on/off status of the first fan 130, etc., in response to control from the control unit 126.

The mask 100 includes the first air inflow path 112, along which air enters the mask 100 through a first air inlet 136a and travels to the first fan 130, a first connecting air flow path along which air travels from the first fan 130 to the first heat sink 134, and the first air outflow flow path 114 along which air travels from the first heat sink 134 and exits the mask 100 through first and second air outlets 138a, 138b. As shown in FIGS. 8-10, the first air inflow path 112 is defined by first ducting 113 extending between the first air inlet 136a and the first fan 130. The first ducting 113 is formed by the intermediate shell 110. As shown in FIG. 8, the first air outflow path 114 is defined by second ducting 115 extending from the first heat sink 134 toward the first and second air outlets 138a, 138b. The second ducting 115 is formed by the intermediate shell 110. The first air inflow path 112, the first connecting air flow path, and the first air outflow path 114 thus define a first air flow path through the mask 100 from the first air inlet 136a to first and second air outlets 138a, 138b. The first airflow path is U-shaped, with air entering the first airflow path through the first air inlet 136a, then flowing upward to the first fan 130, then flowing laterally (inward, to the right, for the first airflow path on the left side of the mask 100) to the first heat sink 134, and then flowing downward before exiting the mask 100 through the first air outlet 138a. The U shape is upside down, e.g., the free ends of the legs of the U face downward.

Similarly, the mask 100 includes the second air inflow path, along which air enters the mask 100 through a second air inlet 136b and travels to the second fan, a second connecting air flow path along which air travels from the second fan to the second heat sink, and the second air outflow flow path along which air travels from the second heat sink and exits the mask 100 through the first and second air outlets 138a, 138b. The second air inflow path is defined by third ducting extending between the second air inlet 136b and the second fan. The third ducting is formed by the intermediate shell 110. The second air outflow path is defined by fourth ducting extending from the second heat sink toward the first and second air outlets 138a, 138b. The fourth ducting is formed by the intermediate shell 110. The second air inflow path, the second connecting air flow path, and the second air outflow path thus define an air flow path through the mask 100 from the second air inlet 136b to the first and second air outlets 138a, 138b. The second airflow path is U-shaped, with air entering the second airflow path through the second air inlet 136b, then flowing upward to the second fan, then flowing laterally (inward, to the left, for the second airflow path on the right side of the mask 100) to the second heat sink, and then flowing downward before exiting the mask 100 through the second air outlet 138b. The U shape is upside down, e.g., the free ends of the legs of the U face downward.

Because the intermediate shell 110 that forms the first and second ducting 113, 115 and the third and fourth ducting is located between the outer and inner shells 106, 108 and because a perimeter of the base 102 of the mask 100 is sealed, so as to be airtight, except at the first and second air inlets 136a, 136b and the first and second air outlets 138a, 138b, air can only flow in the mask 100 along the first and second air flow paths, which may help maximize heat dissipation.

The first and second air outlets 138a, 138b are shared air outlets for the first and second air flow paths. Thus, in this illustrated implementation, the U-shaped first and second air flow paths have some overlap in the downward air flow portions of the first and second air flow paths, and the first and second air flow paths together define a W shape. The W is upside down, e.g., the free ends of the legs of the W face downward. In some implementations, there is a different number of shared air outlets, e.g., one, three, etc. In some implementations, the mask 100 has separate air outlets for each of the first and second air flow paths.

The first and second air inlets 136a, 136b and the first and second air outlets 138a, 138b are located at a bottom of the mask 100, as shown in FIGS. 2-4 and 16. Thus, as indicated by FIG. 1, the first and second air inlets 136a, 136b and the first and second air outlets 138a, 138b are not visible when looking at a front of the mask 100, which may improve aesthetics of the mask 100. In other implementations, any one or more of the first and second air inlets 136a, 136b and the first and second air outlets 138a, 138b can be located elsewhere, e.g., at a top of the mask 100 or a side of the mask 100, instead of at the bottom of the mask 100 and may thus similarly not be visible when looking at a front of the mask 100.

As discussed herein, the running of the first and second fans 130, e.g., when the fans 130 are on and causing air flow along the first and second air flow paths, creates noise that can irritate the user wearing the mask 100. The mask 100 includes first and second noise attenuation systems associated with the first and second fans 130, respectively. The first and second noise attenuation systems are configured to attenuate the generated noise that is radiated into the environment due to use of the first and second fans 130, respectively. The first and second noise attenuation systems are configured and used similarly so are not each particularly described, with features described for the first noise attenuation system similarly applying to the second noise attenuation system.

The first and second noise attenuation systems can have a variety of configurations. Various exemplary implementations of noise attenuation systems that can be used with the mask 100 and other implementations of masks described herein are described further in, for example, previously mentioned U.S. Pat. patent application Ser. No. 18/411,644 entitled “Face Masks With Noise Attenuation” filed Jan. 12, 2024 and U.S. Pat. Pub. No. 2024/0257792 entitled “Acoustic Muffler For A Motorized Food Processing Device” published Aug. 1, 2024.

In this illustrated implementation, as shown in FIGS. 8-10, the first noise attenuation system includes a first acoustic chamber 140 upstream of its associated fan 130. The acoustic chamber 140 is generally configured as a silencer configured to muffle sound. The air entering the first air inlet 136a and flowing along the first air inflow path causes noise that would be easily heard by the user without the first acoustic chamber 140 being present to attenuate the noise so as to reduce, if not eliminate, bothersome noise to the user causes by the air inflow.

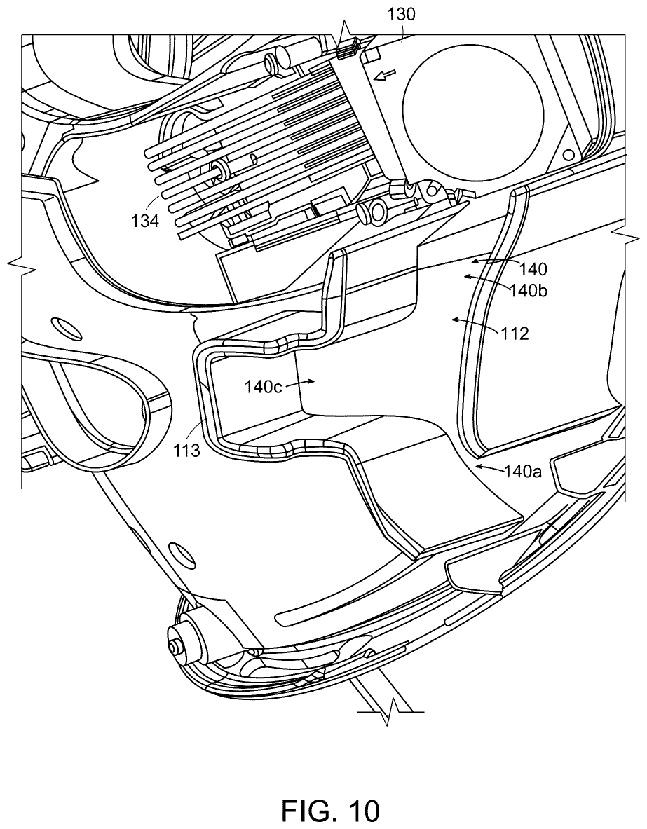

The first acoustic chamber 140 is located adjacent the first air inlet 136a. Air is thus configured to enter the first air inlet 136a and flow into the first acoustic chamber 140. An outlet end of the first acoustic chamber 140 is open to allow acoustic energy to exit the first acoustic chamber 140 in a direction toward the first fan 130. As shown in FIG. 10, the first acoustic chamber 140 includes an inlet chamber 140a, an outlet chamber 140b, and a central chamber 140c that is located between and is in fluid communication with the inlet and outlet chambers 140a, 140b. In this illustrated implementation, the central chamber 140c expands laterally to only one side in relation to the inlet and outlet chambers 140a, 140b. The central chamber 140c is expanded laterally inward, but the lateral expansion could instead be laterally outward.

As shown in FIG. 15, the first cooling system of the mask 100 also includes a temperature sensor 142 and a thermal cutoff (TCO) 144. The temperature sensor 142 is a negative temperature coefficient (NTC) temperature sensor in this illustrated implementation but can be another type of temperature sensor. In general, the temperature sensor 142 and the TCO 144 are configured to help ensure that safe, comfortable, therapeutic cooling is provided to the user via the mask 100.

The temperature sensor 142 is configured to measure a temperature of the first coolsink 127 (first pad 127). The temperature sensor 142 is operatively coupled to the PCB 125 of the mask 100 and is configured to provide the measured temperature to the PCB 125 of the mask 100. The PCB 125 of the mask 100 is operably coupled to the PCB 126b of the control unit 126 and is configured to provide the measured temperature to the PCB 126b of the control unit 126.

The control unit 126 is configured to compare the measured temperature of the first coolsink 127 with a predetermined temperature associated with a current cooling mode. If the measured temperature equals the predetermined temperature, then appropriate cooling is being provided to the user via the first coolsink 127 (either directly or through the second coolsink 129). The control unit 126 takes no further action until a next measured temperature is received.

If the measured temperature does not equal the predetermined temperature, then appropriate cooling is not being provided to the user via the first coolsink 127 (either directly or through the second coolsink 129). In response to the measured temperature being greater than the predetermined temperature, the control unit 126 is configured to trigger a corrective action to reduce temperature of the first coolsink 127. For example, the control unit 126 can cause the first fan 130 to increase power to blow more air across the heatsink 134 to dissipate more heat, and/or the control unit 126 can cause less voltage to be provided to the first coolsink 127 to reduce temperature of the first coolsink 127. In response to the measured temperature being less than the predetermined temperature, the control unit 126 is configured to trigger a corrective action to increase temperature of the first coolsink 127. For example, the control unit 126 can cause the first fan 130 to decrease power to blow more air across the heatsink 134 to dissipate less heat and/or the control unit 126 can cause more voltage to be provided to the first coolsink 127 to increase temperature of the first coolsink 127.

Determining if the measured temperature equals the predetermined temperature is, in some implementations, an exact match and is, in other implementations, a match within a +/− margin of acceptable tolerance.

In an exemplary implementation, the control unit 126, e.g., the PCB 126b of the control unit 126, is configured to receive temperature data measured by the temperature sensor 142 (and similar temperature sensor for the second cooling system) on a regular, periodic basis throughout the mask 100 providing cooling therapy. The control unit 126 may thus be able to cause corrective action to be performed as needed throughout the cooling therapy.

In some implementations, instead of the control unit 126, e.g., the PCB 126b of the control unit 126, being configured to compare the measured temperature of the first coolsink 127 with a predetermined temperature associated with a current cooling mode, the mask 100, e.g., the PCB 125 of the mask 100, is configured to perform the comparison. If the measured temperature equals the predetermined temperature, the PCB 125 of the mask 100 takes no further action until a next measured temperature is received. If the measured temperature does not equal the predetermined temperature, the PCB 125 of the mask 100 communicates a temperature error to the control unit 126, e.g., the PCB 126b of the control unit 126, so the control unit 126 can cause appropriate corrective action, as discussed above.

The TCO 144 is configured as a safety device to interrupt electrical power to the first coolsink 127 in response to the temperature of the first coolsink 127 being above a maximum predetermined temperature. Interrupting the electrical power to the first coolsink 127 causes the first coolsink 127 to stop generating heat and cold. The first coolsink 127 will not typically reach a temperature above the maximum predetermined temperature, but in rare instance of malfunction, the TCO 144 may help prevent overheating of the first cooling system and/or an unsafe and/or uncomfortable temperature being provided against the user's skin.

Each of the first and second cooling systems including a temperature sensor and a TCO allows for independent control of the first and second cooling systems, which may improve accuracy of cooling therapy provided to the user.

In an exemplary implementation, the control unit 126 is configured to allow a user to select one of a plurality of cooling modes, e.g., by using the third control 126f to scroll through available cooling modes shown on the display 126c and select a desired one of the cooling modes. As mentioned above, each of the plurality of cooling modes is associated with a predetermined temperature to be applied to the user's skin via the first set of pads 127 (and, at the user's option, also the second set of pads 129). The predetermined temperature for each of the plurality of cooling modes is stored in a memory at the PCB 126b of the control unit 126 and/or in a memory at the PCB 125 of the mask 100.

In an exemplary implementation, the plurality of cooling modes are each associated with a different predetermined temperature that is 25° C. or less and that is at least 3° C. above/below adjacent mode temperatures. Temperatures less than 25° C. will cause vasoconstriction that results in depuffing. A temperature difference of at least 3° C. will typically be felt by a user whereas a lower temperature difference will not typically be felt by a user, so having temperatures of cooling modes each being at least 3° C. apart will allow the user to feel a difference between each of the cooling modes and thus help the user choose a comfortable cooling mode and/or know that the cooling modes are different from one another. For example, the plurality of cooling modes can include first, second, and third cooling modes each associated with a different predetermined temperature, e.g., 16° C., 19° C., and 22° C. For another example, the plurality of cooling modes can include first, second, third, and fourth cooling modes each associated with a different predetermined temperature, e.g., 16° C., 19° C., 22° C., and 25° C. For another example, the plurality of cooling modes can include first, second, and third cooling modes each associated with a different predetermined temperature, e.g., 19° C., 22° C., and 25° C. For another example, the plurality of cooling modes can include first, second, and third cooling modes each associated with a different predetermined temperature, e.g., 15° C., 19° C., and 23° C. For another example, the plurality of cooling modes can include first and second cooling modes each associated with a different predetermined temperature, e.g., 16° C. and 22° C. For another example, the plurality of cooling modes can include first and second cooling modes each associated with a different predetermined temperature, e.g., 19° C. and 22° C. For another example, the plurality of cooling modes can include first and second cooling modes each associated with a different predetermined temperature, e.g., 16° C. and 19° C. For another example, the plurality of cooling modes can include first and second cooling modes each associated with a different predetermined temperature, e.g., 19° C. and 25° C. For another example, the plurality of cooling modes can include first and second cooling modes each associated with a different predetermined temperature, e.g., 15° C. and 19° C. For another example, the plurality of cooling modes can include first and second cooling modes each associated with a different predetermined temperature, e.g., 19° C. and 22° C. For another example, the plurality of cooling modes can include first and second cooling modes each associated with a different temperature in a range of 15° C. to 25° C.

The selected cooling mode can, in some implementations, be changed during the cooling therapy, which may provide the user greater control over cooling therapy.

In an exemplary implementation, whether or not the control unit 126 is configured to allow a user to select one of a plurality of cooling modes, the control unit 126 is configured to allow a user to select a time duration of cooling from among a plurality of time durations. In general, a longer duration of cooling may cause greater therapeutic effect and/or may help treat more stubborn problems. The plurality of time durations can be, for example, any two or more of five minutes, six minutes, seven minutes, eight minutes, nine minutes, ten minutes, twelve minutes, thirteen minutes, fourteen minutes, fifteen minutes, sixteen minutes, seventeen minutes, eighteen minutes, nineteen minutes, and twenty minutes. For example, the plurality of time durations can be five minutes apart from one another (e.g., five minutes, ten minutes, and fifteen minutes). A user will typically be able to tell differences between such staggered durations whereas shorter durations are typically more different to detect.

In instances in which a user chooses to receive light therapy and cooling therapy simultaneously, the duration of the cooling therapy can be defined by a duration of the selected light therapy instead of being independently selectable, which may ease user experience by not requiring the user to select multiple different times. Cooling therapy duration is not desirable to define light therapy duration since a maximum amount of time cooling therapy is provided is typically less than a maximum amount of time light therapy is provided, e.g., a maximum of fifteen minutes for cooling therapy and a maximum of eight minutes for light therapy. In some implementations, a user may choose both light therapy duration and cooling therapy duration.

Various exemplary implementations of modes for a face mask are described further in, for example, U.S. patent application Ser. No. 18/901,465 entitled “Therapeutic Face Mask Modes Of Operation” filed on Sep. 30, 2024, which is hereby incorporated by reference in its entirety.

As mentioned above, the control unit 126 includes a rechargeable power source 126a in this illustrated implementation. The power source 126a can be recharged in any of a variety of ways. In an exemplary implementation, the control unit 126 is configured to be docked in a charging stand for recharging. The charging stand also serves as a storage stand for the control unit 126 and the mask 100. Recharging may therefore occur conveniently at the same location where the control unit 126 and the mask 100 are being stored between uses. The charging stand can have a variety of configurations.

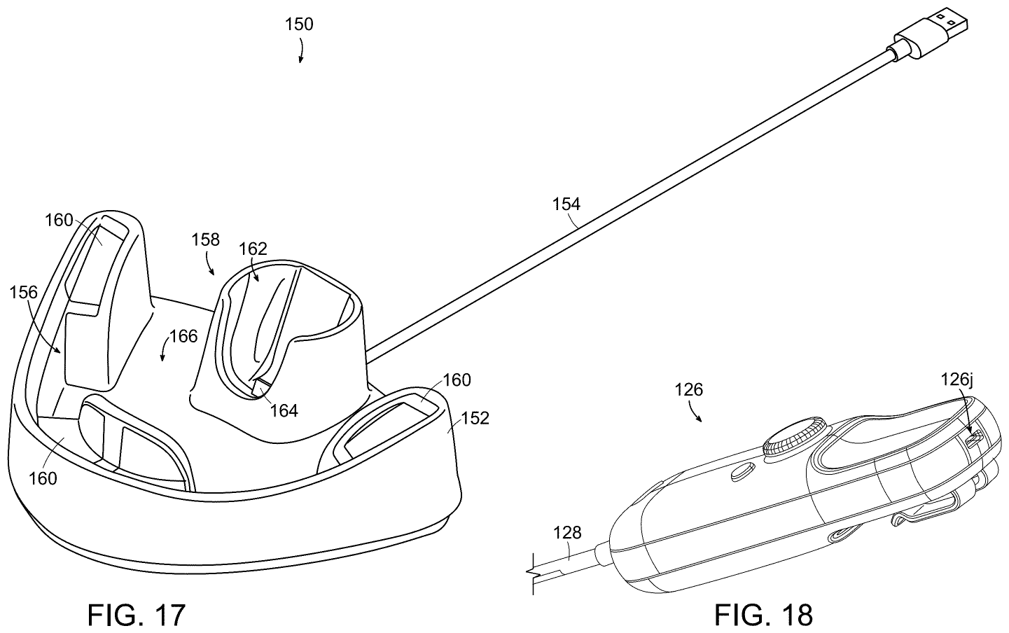

FIG. 17 illustrates one exemplary implementation of charging stand 150. The illustrated charging stand 150 is sized and shaped for use with the mask 100 and the control unit 126 of FIG. 1. Other charging stands having different sizes and/or shapes can be used similarly with other masks and control units.

The charging stand 150 includes a base 152 and a power cord 154. The power cord 154 is configured to operably connect to a power source to provide power for recharging. The power cord 154 in this illustrated implementation is a USB cord configured to be plugged into a USB port, but other types of power cords can be used, such as a cord configured to be plugged into a wall outlet.

The base 152 is configured to dock the mask 100 and the control unit 126. The base 152 includes a first docking area 156 configured to dock the mask 100 and a second docking area 158 configured to dock the control unit 126. The first docking area 156 includes one or more mask supports 160 configured to seat the mask 100. The one or more mask supports 160 are configured to prevent the mask 100 from tipping over or falling off the stand 150. The bottom of the mask 100 is configured to rest on the one or more mask supports 160 in this illustrated implementation. The mask supports 160 includes three support in this illustrated implementation but another number of mask supports 160 may be provided.

The second docking area 158 defines a cavity 162 configured to receive the control unit 126 therein. A charger interface 164 is located within the cavity 162. The charger interface 164 is configured to be received in a charging port 126j of the control unit 126 (see FIG. 18). With the power cord 154 attached to a power source, e.g., plugged into a USB port, and with the charger interface 164 received in the charging port 126j, power is configured to be provided to the control unit 126 via the connected charger interface 164 and charging port 126j to recharge the power source 126a of the control unit 126. Recharging is configured to occur automatically with the power cord 154 attached to a power source and with the charger interface 164 received in the charging port 126j, which may help ensure that recharging occurs regularly since the mask 100 and the control unit 126 will typically be stored in the stand 150 when the mask 100 is not being worn by a user. Alternatively, the charging stand 150 and/or the control unit 126 can include a recharging control configured to allow a user to turn on/off recharging.

The charging stand 150 includes a storage area 166 between the first and second docking areas 156, 158 that defines space configured to accommodate the cable 128 while the mask 100 and the control unit 126 are docked. A user may bend the cable 128 as needed to fit the cable 128 into the storage area 166.

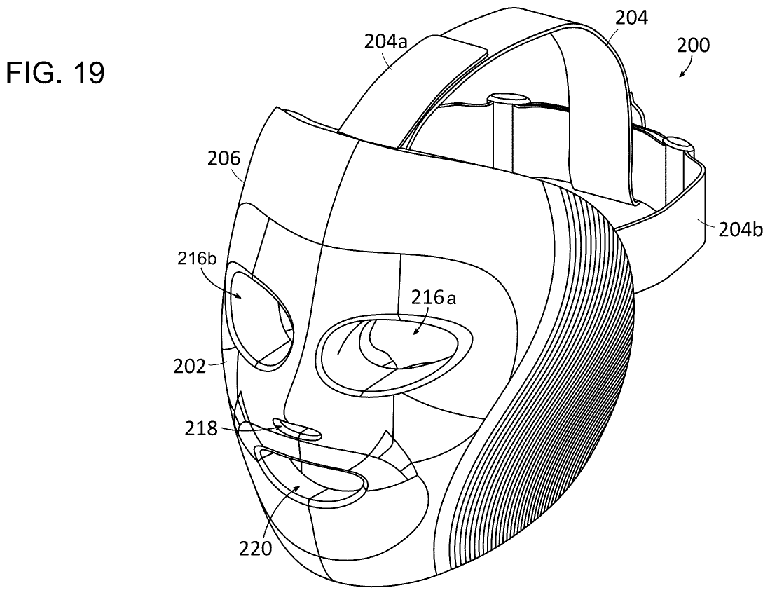

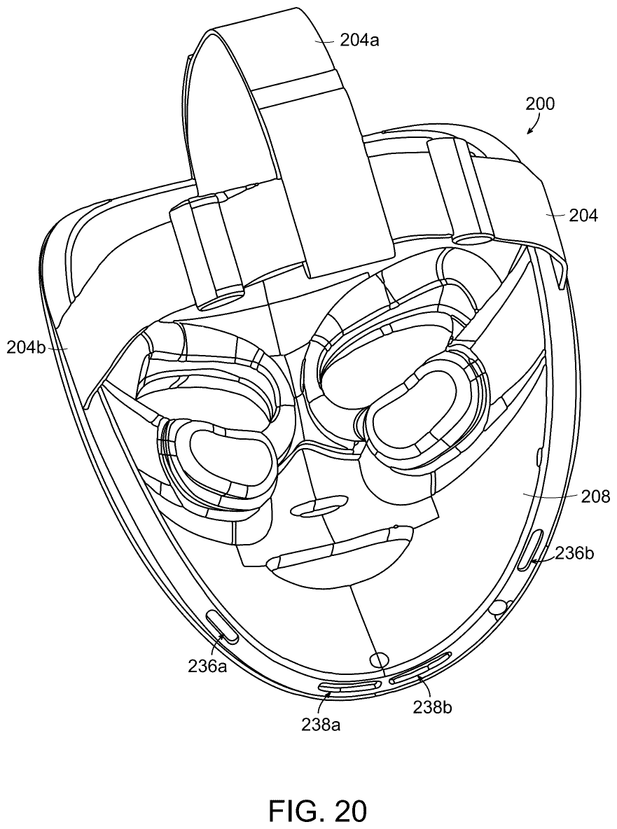

FIGS. 19 and 20 illustrate another implementation of a face mask 200 configured to provide cooling therapy to a user wearing the face mask 200. The face mask 200 in this illustrated implementation is also configured to provide light therapy.

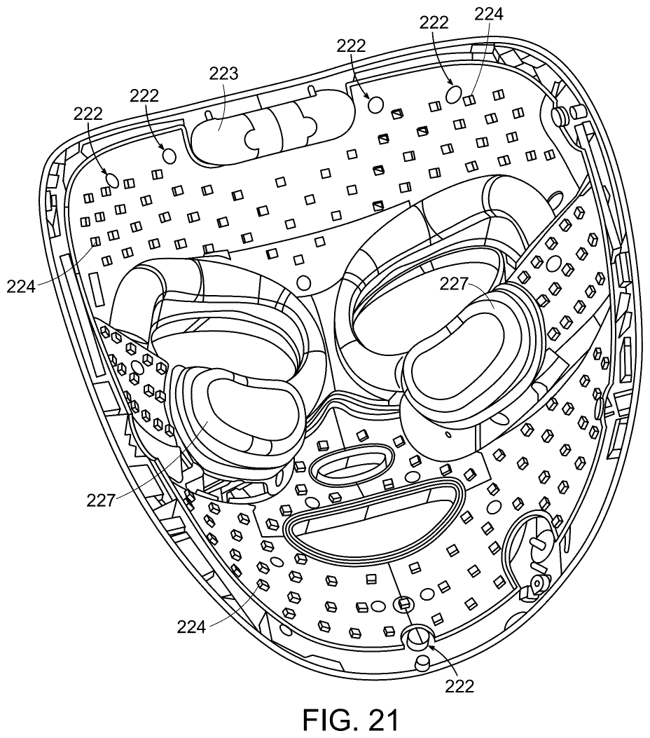

The face mask 200 of FIGS. 19 and 20 is generally configured and used similar to the face mask 100 of FIG. 1 and includes a base 202 including an outer shell 206 (see FIG. 19), an inner shell 208 (see FIG. 20), and an intermediate shell 210 (see FIGS. 21 and 22); a support 204 including first and second straps 204a, 204b; first and second air inflow paths (obscured in the figures), first and second air outflow paths (obscured in the figures); a plurality of openings including a first eye opening 216a, a second eye opening 216b, a nose opening 218, and a mouth opening 220; cut-outs 222 at a plurality of interior connection points; a light assembly including a plurality of lights 224; a PCB (obscured in the figures); first and second cooling systems each including a fan (obscured in the figures), a thermoelectric cooling device (first and second thermoelectric cooling devices 232a, 232b shown in FIG. 22), and a heat sink (obscured in the figures); a forehead pad 223; first and second eye shields 225a, 225b; a set of eye pads 227, first and second air inlets 236a, 236b; first and second air outlets 238a, 238b; and first and second noise attenuation systems (obscured in the figures). The mask 200 can include any one or more of the variations discussed above that the mask 100 of FIG. 1 may have.

FIG. 22 shows the mask 200 without the set of pads 227 attached to the mask 100 for purposes of illustration, since the set of pads 227 are non-removably attached to the mask 200 in this illustrated implementation. The set of pads 227 are the only set of pads in this illustrated embodiment.

A control unit (now shown) is configured to be operably coupled with the mask 200 using a cable (not shown) and is generally configured and used similar to the control unit 126 of FIG. 1. The control unit used with the mask 200 can include any one or more of the variations discussed above that the control unit 126 of FIG. 1 may have.

Various face masks, e.g., the masks 100, 200 of FIGS. 1 and 19 and other masks, are configured to provide under-eye cooling. The masks 100, 200 of FIGS. 1 and 19 each include two thermoelectric cooling devices (the first and second thermoelectric cooling devices 132 of the mask 100 of FIG. 1 and the first and second thermoelectric cooling devices 232a, 232b of the mask 200 of FIG. 19) to allow for one thermoelectric cooling device to be positioned under each eye of a user wearing the mask 100, 200. In some implementations, instead of including only one thermoelectric cooling device to be positioned under each eye of a user wearing the mask 100, 200 (or other implementation of a mask), the mask 100, 200 includes a first plurality of thermoelectric cooling devices configured to be positioned under a left eye of a user wearing the mask and a second plurality of thermoelectric cooling devices configured to be positioned under a right eye of a user wearing the mask. Including multiple thermoelectric cooling devices under each eye may help ensure that at least some cooling is provided under an eye in the unlikely event that a thermoelectric cooling device fails and/or may free mask real estate for one or more lights configured to emit light for light therapy.

In some implementations, in addition to including at least one thermoelectric cooling device configured to be positioned under a left eye of a user wearing a mask, e.g., the masks 100, 200 of FIGS. 1 and 19 and other masks, and at least one thermoelectric cooling device configured to be positioned under a right eye of the user wearing the mask, the mask includes at least one thermoelectric cooling device configured to be positioned at a left temple of the user wearing the mask to provide cooling to the left temple and at least one thermoelectric cooling device configured to be positioned at a right temple of the user wearing the mask to provide cooling to the right temple. Cooling applied to the user's temples is configured to depuff, soothe, and refresh the user's left and right areas by shrinking blood vessels in the left and right temple areas.

As discussed herein, some implementations of masks, e.g., the masks 100, 200 of FIGS. 1 and 19 and other masks, are configured to provide cooling therapy and to provide light therapy. For example, the mask can include at least two thermoelectric cooling devices (located under eyes and/or at temples) configured to provide cooling therapy and a plurality of lights configured to provide light therapy. In some implementations, the mask configured to provide cooling therapy and to provide light therapy includes a first plurality of lights configured to be positioned under a right eye of the user wearing the mask to provide light therapy to the user's right under-eye area and includes a second plurality of lights configured to be positioned under a left eye of the user wearing the mask to provide light therapy to the user's left under-eye area. In an exemplary implementation, the under-eye lights follow a curvature of a lower edge of their respective eyes to help maximize light therapy to the user's left and right under eye areas.

In some implementations, a mask including a first plurality of lights configured to be positioned under a right eye of the user wearing the mask to provide light therapy to the user's right under-eye area and including a second plurality of lights configured to be positioned under a left eye of the user wearing the mask to provide light therapy to the user's left under-eye area is configured to provide light therapy and to not provide cooling therapy.

In some implementations, whether or not a mask is configured to provide cooling therapy, a mask, e.g., the mask 100 of FIG. 1, the mask 200 of FIG. 19, or other mask, includes at least one light configured to be positioned at a right temple of the user wearing the mask to provide light therapy to the user's right temple and includes at least one light configured to be positioned at a left temple of the user wearing the mask to provide light therapy to the user's left temple. The user's left and right temples may thus be more sure to receive effective light therapy.

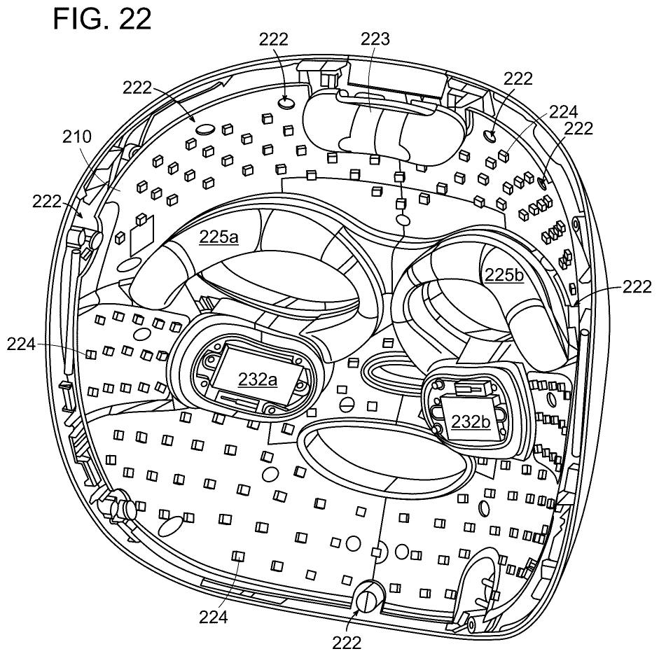

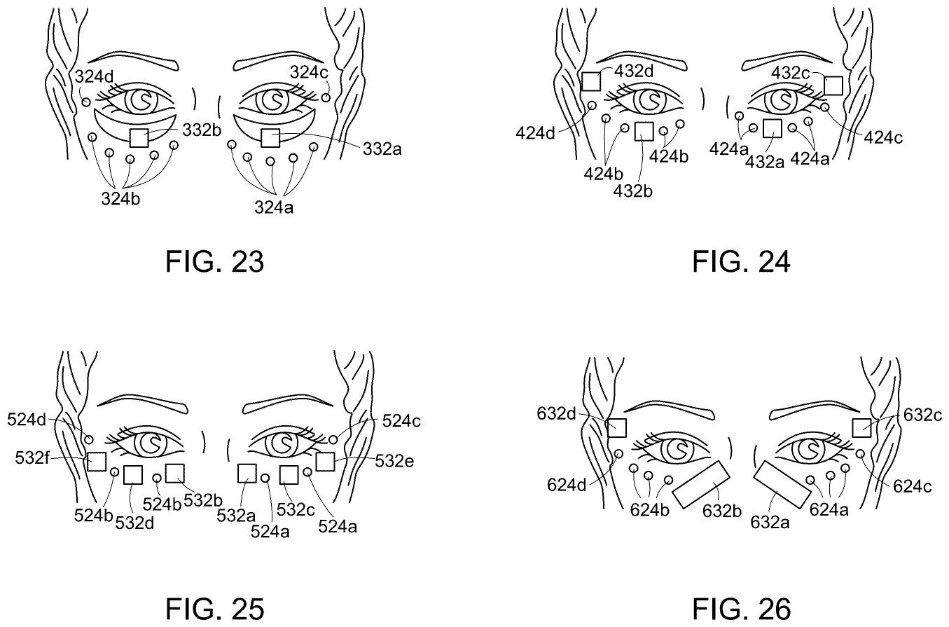

FIG. 23 illustrates one implementation of first and second thermoelectric cooling devices 332a, 332b of a mask (not shown for clarity of illustration), a first plurality of under-eye lights 324a, a second plurality of under-eye lights 324b, at least one left temple light 324c, and at least one right temple light 324d. As shown in FIG. 23, the first and second thermoelectric cooling devices 332a, 332b are configured to be positioned under left and right eyes, respectively, of a user wearing the mask, the first plurality of under-eye lights 324a is configured to be positioned under the user's left eye and to follow a curvature of a lower edge of the left eye, the second plurality of under-eye lights 324b is configured to be positioned under the user's right eye and to follow a curvature of a lower edge of the right eye, the at least one left temple light 324c is configured to be positioned at the user's left temple, and the at least one right temple light 324d is configured to be positioned at the user's right temple. The first and second plurality of under-eye lights 324a, 324b each include five lights in this illustrated implementation, but another number may be used, e.g., two, three, four, six, seven, eight, nine, ten, etc. The at least one left temple light 324c and the at least one right temple light 324d each include one light in this illustrated implementation, but another number may be used, e.g., two, three, etc.

FIG. 24 illustrates one implementation of first, second, third, and fourth thermoelectric cooling devices 432a, 432b, 432c, 432d of a mask (not shown for clarity of illustration), a first plurality of under-eye lights 424a, a second plurality of under-eye lights 424b, at least one left temple light 424c, and at least one right temple light 424d. As shown in FIG. 24, the first and second thermoelectric cooling devices 432a, 432b are configured to be positioned under left and right eyes, respectively, of a user wearing the mask, the third and fourth thermoelectric cooling devices 432c, 432d are configured to be positioned at left and right temples, respectively, of the user, the first plurality of under-eye lights 424a is configured to be positioned under the user's left eye and to follow a curvature of a lower edge of the left eye, the second plurality of under-eye lights 424b is configured to be positioned under the user's right eye and to follow a curvature of a lower edge of the right eye, the at least one left temple light 424c is configured to be positioned at the user's left temple, and the at least one right temple light 424d is configured to be positioned at the user's right temple. The first and second plurality of under-eye lights 424a, 424b each include four lights in this illustrated implementation, but another number may be used, e.g., two, three, five, six, seven, eight, nine, ten, etc. The at least one left temple light 424c and the at least one right temple light 424d each include one light in this illustrated implementation, but another number may be used, e.g., two, three, etc.

FIG. 25 illustrates one implementation of first, second, third, fourth, fifth, and sixth thermoelectric cooling devices 532a, 532b, 532c, 532d, 532e, 532f of a mask (not shown for clarity of illustration), a first plurality of under-eye lights 524a, a second plurality of under-eye lights 524b, at least one left temple light 524c, and at least one right temple light 524d. As shown in FIG. 25, the first, third, and fifth thermoelectric cooling devices 532a, 532c, 532e are configured to be positioned under a left eye of a user wearing the mask, the second, fourth, and sixth thermoelectric cooling devices 532b, 532d, 532f are configured to be positioned under a right eye of a user wearing the mask, the first plurality of under-eye lights 524a is configured to be positioned under the user's left eye and to follow a curvature of a lower edge of the left eye, the second plurality of under-eye lights 524b is configured to be positioned under the user's right eye and to follow a curvature of a lower edge of the right eye, the at least one left temple light 524c is configured to be positioned at the user's left temple, and the at least one right temple light 524d is configured to be positioned at the user's right temple. The first and second plurality of under-eye lights 524a, 524b each include two lights in this illustrated implementation, but another number may be used, e.g., three, four, five, six, seven, eight, nine, ten, etc. The at least one left temple light 524c and the at least one right temple light 524d each include one light in this illustrated implementation, but another number may be used, e.g., two, three, etc.

FIG. 26 illustrates one implementation of first, second, third, and fourth thermoelectric cooling devices 632a, 632b, 632c, 632d of a mask (not shown for clarity of illustration), a first plurality of under-eye lights 624a, a second plurality of under-eye lights 624b, at least one left temple light 624c, and at least one right temple light 624d. As shown in FIG. 26, the first and second thermoelectric cooling devices 632a, 632b are configured to be positioned under left and right eyes, respectively, of a user wearing the mask, the third and fourth thermoelectric cooling devices 632c, 632d are configured to be positioned at left and right temples, respectively, of the user, the first plurality of under-eye lights 624a is configured to be positioned under the user's left eye and to follow a curvature of a lower edge of the left eye, the second plurality of under-eye lights 624b is configured to be positioned under the user's right eye and to follow a curvature of a lower edge of the right eye, the at least one left temple light 624c is configured to be positioned at the user's left temple, and the at least one right temple light 624d is configured to be positioned at the user's right temple. The first and second plurality of under-eye lights 624a, 624b each include three lights in this illustrated implementation, but another number may be used, e.g., two, four, five, six, seven, eight, nine, ten, etc. The at least one left temple light 624c and the at least one right temple light 624d each include one light in this illustrated implementation, but another number may be used, e.g., two, three, etc.

The subject matter described herein can be implemented in analog electronic circuitry, digital electronic circuitry, and/or in computer software, firmware, or hardware, including the structural means disclosed in this specification and structural equivalents thereof or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, algorithm, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code).

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices). The processor and the memory can be supplemented by, or incorporated in, special purpose logic circuitry.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor-readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor). The modules described herein can be combined, integrated, separated, and/or duplicated to support various applications. Also, a function described herein as being performed at a particular module can be performed at one or more other modules and/or by one or more other devices instead of or in addition to the function performed at the particular module.

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A therapeutic device, comprising:
a face mask configured to be worn on a face of a user, the face mask comprising:
a first thermoelectric cooling device configured to be positioned under a first eye of the user, configured to generate cooling configured to be applied to skin under the first eye of the user, and configured to generate heat,
a second thermoelectric cooling device configured to be positioned under a second eye of the user, configured to generate cooling configured to be applied to skin under the second eye of the user, and configured to generate heat, and
a plurality of light emitting diodes (LEDs) configured to emit light toward and to skin on the face of the user with the user wearing the face mask.

2. The therapeutic device of claim 1, wherein the first and second thermoelectric cooling devices are each a Peltier device.

3. The therapeutic device of claim 1, wherein the LEDs are each configured to emit different wavelengths of light.

4. The therapeutic device of claim 1, wherein:
the face mask further comprises:
a first fan upstream of the first thermoelectric cooling device and configured to cause air flow in the face mask along a first air flow path to dissipate the heat generated by the first thermoelectric cooling device,
a first heat sink located along the first air flow path downstream of the first fan,
a second fan upstream of the second thermoelectric cooling device and configured to cause air flow in the face mask along a second air flow path to dissipate the heat generated by the second thermoelectric cooling device, and
a second heat sink located along the second air flow path downstream of the second fan.

5. The therapeutic device of claim 1, further comprising a control unit operably coupled to the face mask;
wherein the control unit is configured to control the plurality of lights, the cooling configured to be applied to skin under the first eye of the user, and the cooling configured to be applied to skin under the second eye of the user.

6. A therapeutic device, comprising:
a face mask configured to be worn on a face of a user, the face mask comprising:
a first thermoelectric cooling device configured to be positioned under a first eye of the user, configured to generate cooling configured to be applied to skin under the first eye of the user, and configured to generate heat,
a first air inlet through which air external to the face mask is configured to enter a U-shaped first air flow path in the face mask,
a first air outlet through which the air is configured to exit the first air flow path to exit the face mask,
a first fan upstream of the first thermoelectric cooling device and configured to cause the air to enter the face mask through the first air inlet and to flow along the first air flow path, the air flowing along the first air flow path being configured to dissipate the heat generated by the first thermoelectric cooling device,
a second thermoelectric cooling device configured to be positioned under a second eye of the user, configured to generate cooling configured to be applied to skin under the second eye of the user, and configured to generate heat,
a second air inlet through which air external to the face mask is configured to enter a U-shaped second air flow path in the face mask,
a second air outlet through which the air is configured to exit the second air flow path to exit the face mask, and
a second fan upstream of the second thermoelectric cooling device and configured to cause the air to enter the face mask through the second air inlet and to flow along the second air flow path, the air flowing along the second air flow path being configured to dissipate the heat generated by the second thermoelectric cooling device.

7. The therapeutic device of claim 6, wherein the first and second thermoelectric cooling devices are each a Peltier device.

8. The therapeutic device of claim 6, wherein the first air inlet, the first air outlet, the second air inlet, and the second air outlet are each located at a bottom of the face mask.

9. The therapeutic device of claim 6, wherein the face mask further comprises:
a first heat sink located along the first air flow path downstream of the first fan, and
a second heat sink located along the second air flow path downstream of the second fan;
the air entering the first air inlet is configured to flow to the first fan and then laterally inward to the first heat sink; and
the air entering the second air inlet is configured to flow to the second fan and then laterally inward to the second heat sink.

10. The therapeutic device of claim 9, wherein the air flowing laterally inward to the first heat sink is in an opposite direction to the air flowing laterally inward to the second heat sink.

11. The therapeutic device of claim 9, wherein the face mask further comprises:
first internal ducting extending between the first air inlet and the first fan and extending from the first heat sink toward the first air outlet, and
second internal ducting extending between the second air inlet and the second fan and extending from the second heat sink toward the second air outlet.

12. The therapeutic device of claim 6, wherein the face mask is air tight around a perimeter of the face mask except at the first air inlet, the first air outlet, the second air inlet, and the second air outlet.

13. The therapeutic device of claim 6, wherein the face mask further comprises an outer shell, an inner shell, and an intermediate shell located between the outer and inner shell; and
the intermediate shell defines first ducting along the first air flow path and defines second ducting along the second air flow path.

14. The therapeutic device of claim 13, wherein the first ducting comprises first inflow ducting extending between the first air inlet and the first fan and first outflow ducting extending toward the first air outlet; and
the second ducting comprises second inflow ducting extending between the second air inlet and the second fan and second outflow ducting extending toward the second air outlet.

15. The therapeutic device of claim 6, wherein the air flowing along the first air flow path is also configured to exit the face mask through the second air outlet; and
the air flowing along the second air flow path is also configured to exit the face mask through the first air outlet.

16. The therapeutic device of claim 6, wherein the first and second air flow paths together define a W shape.

17. The therapeutic device of claim 6, wherein the cooling configured to be applied to skin under the first eye of the user and the cooling configured to be applied to skin under the second eye of the user are each configured to be at a temperature in a range of 15° C. to 25° C.

18. The therapeutic device of claim 6, further comprising a control unit operably coupled to the face mask;
wherein the control unit is configured to receive an input from the user and to control, based on the input, the cooling configured to be applied to skin under the first eye of the user and the cooling configured to be applied to skin under the second eye of the user.

19. The therapeutic device of claim 6, wherein the face mask further comprises a plurality of light emitting diodes (LEDs) configured to emit light toward and to skin on the face of the user with the user wearing the face mask.

20. The therapeutic device of claim 19, further comprising a control unit operably coupled to the face mask;
wherein the control unit is configured to control the plurality of lights, the cooling configured to be applied to skin under the first eye of the user, and the cooling configured to be applied to skin under the second eye of the user.
